(19) United States
(12) Patent Application Publication
(10) Pub. No.: US 2020/0163998 A1
(43) Pub. Date: May 28, 2020

(54) NANOVESICLES PRODUCED FROM MESENCHYMAL STROMAL CELLS FOR ANTI-INFLAMMATORY APPLICATIONS

(71) Applicant: Codiak BioSciences, Inc., Cambridge, MA (US)

(72) Inventors: Kyong-su PARK, Cambridge, MA (US); Elga Bernardo BANDEIRA DE MELO, Cambridge, MA (US); Jan LOTVALL, Cambridge, MA (US)

(73) Assignee: Codiak BioSciences, Inc., Cambridge, MA (US)

(21) Appl. No.: 16/632,122

(22) PCT Filed: Jul. 17, 2018
(86) PCT No.: PCT/US2018/042409

§ 371 Date: Jan. 17, 2020

Related U.S. Application Data
(60) Provisional application No. 62/533,629, filed on Jul. 17, 2017.

(51) Int. Cl. A61K 35/28; A61K 38/17; A61K 31/7105; A61K 38/20; A61K 9/00

(52) U.S. Cl. CPC A61K 35/28; A61K 38/1709; A61K 45/06; A61K 38/2066; A61K 9/0019; A61K 31/7105

(57) ABSTRACT

The invention relates to methods for production of extracellular vesicle-mimetic nanovesicles from mesenchymal stem cells to treat or prevent inflammatory related conditions or diseases. Included with the invention are pharmaceutical compositions comprising extracellular vesicle-mimetic nanovesicles derived from mesenchymal stem cells for the treatment or prevention of inflammatory related conditions.

### CROSS REFERENCE TO RELATED APPLICATIONS

This application claims the benefit of U.S. Provisional Application No. 62/533,629 filed Jul. 17, 2017; which is hereby incorporated by reference in its entirety.

### BACKGROUND OF THE INVENTION

### Field of the Invention

The invention relates to methods and compositions for production of exosome-mimetic nanovesicles (NVs) from mesenchymal stem cells (MSCs) to treat or prevent inflammatory related conditions or diseases. Included with the invention are pharmaceutical compositions comprising NVs derived from MCSs.

### Description of the Related Art

MSCs are a subset of adult stem cells that promote tissue regeneration through paracrine and endocrine effects. Cell therapies with MSCs have been rapidly developed for a wide range of clinical applications, mostly due to the immunomodulation abilities of these cells (J Cell Physiol. 2015 November; 230(11):2606-17).

Extracellular vesicles (EVs) are membrane vesicles with diameters of 40-1000 nm that can mediate local and systemic cell-to-cell communications through the transfer of proteins and nucleic acids (Stem Cells Int. 2017; 2017:1758139). Recently, EVs have been described as important mediators of MSCs effects. Studies have shown that MSCs-derived EVs can improve recovery in lung injury, cardiac injury, and immune diseases, thus they are the focus of increasing attention as therapeutic agents (Expert Opin Biol Ther. 2016 July; 16(7):859-71). Nevertheless, techniques for large-scale EVs production from MSCs are challenging due to the low production of vesicles by these cells and difficulties to achieve purity. Thus, novel methods for the production of vesicles harboring the immunomodulatory abilities of MSCs are needed.

### SUMMARY OF THE INVENTION

In certain aspects, disclosed herein are pharmaceutical compositions for the prevention or treatment of inflammatory-related conditions comprising extracellular vesicle mimetic nanovesicles (NVs) derived from mesenchymal stem cells (MSCs) produced by the process of passing a suspension of MSCs through a plurality of membrane filters. In certain embodiments, the membrane filters comprise pores with diameters of 10 μm or less. In certain embodiments, the NVs have a diameter of between about 20 nm and 250 nm. In some embodiments, the NVs have a substantial amount of nucleic acid within the NVs. In some embodiments, the nucleic acid within the NVs is RNA. In certain embodiments, the MSCs are mammalian. In certain embodiments, the MSCs are cultured in vitro. In certain embodiments, the MSCs are primary cells. In certain embodiments, the MSCs are derived from an autologous donor.

In certain embodiments, the inflammatory related condition is selected from the group consisting of asthma, sepsis, infection, Rheumatoid arthritis, ulcerative colitis, Crohn's disease, tuberculosis, hepatitis, sinusitis, autoimmune disease, inflammatory bowel disease, pelvic inflammatory disease, ulcers, atherosclerosis, erythema, necrosis, vasculitis, ankylosing spondylitis, connective tissue disease, kidney disease, sarcoidosis, thyroiditis, osteoarthritis, Rheumatism, chronic inflammatory demyelinating polyneuropathy, pancreatitis, psoriatic arthritis, periodontitis, Behcet's disease, sinusitis, polymyalgia rheumatic, nephritis, diverticulitis, granulomatosis with polyangilitis, granuloma, encephalitis, immune-mediated inflammatory disease, esophagitis, gout, uveitis, myopathy, gallbladder disease, periodic fever syndrome, interstitial cystitis, peritonitis, appendicitis, Parkinson's disease, Alzheimer's, systemic lupus erythematous, fibromyalgia, diverticulitis, dermatitis and ankylosing spondylitis. In certain embodiments, the inflammatory related condition is asthma. In certain embodiments, the inflammatory related condition is sepsis. In certain embodiments, the inflammatory related condition is infection. In certain embodiments, the infection is a bacterial, viral or parasitic infection.

In certain embodiments, inflammation in at least one tissue in the subject is reduced upon administration of the pharmaceutical composition to a subject. In certain embodiments, the NVs are immunomodulatory when administered intravenously or non-parenterally to a human subject. In certain embodiments, upon administration of the pharmaceutical composition, infiltration of T lymphocytes in lung tissue in reduced compared to administration of a pharmaceutical composition comprising the same concentration of exosomes derived from MSCs.

In certain embodiments, the pharmaceutical composition further comprises a therapeutic payload. In certain embodiments, the therapeutic payload is an inhibitor of Myeloid Differentiation Factor 88 (Myd88). In certain embodiments, the inhibitor of Myd88 is a Myd88 synthetic peptide. In some embodiments, the therapeutic payload is an inhibitor of GATA-3. In certain embodiments, the therapeutic payload comprises nucleic acid. In certain embodiments, the therapeutic payload comprises RNA. In certain embodiments, the therapeutic payload comprises a small-molecule inhibitor. In certain embodiments, the therapeutic payload comprises an antibody.

In certain embodiments, the pharmaceutical composition further comprises a targeting receiver. In certain embodiments, the targeting receiver comprises a protein or peptide.

In certain embodiments, the pharmaceutical composition further comprises Interleukin 10 (IL-10). In certain embodiments, the NVs comprise IL-10. In certain embodiments, the IL-10 is displayed on the surface of the NVs.

In certain embodiments, the pharmaceutical composition is administered in conjunction with at least one additional therapeutic agent. In certain embodiments, the pharmaceutical composition is administered prior to the at least one additional therapeutic agent. In certain embodiments, the pharmaceutical composition is administered after the at least one additional therapeutic agent. In certain embodiments, the pharmaceutical composition is administered simultaneously with the least one additional therapeutic agent. In certain embodiments, the pharmaceutical composition is administered intravenously. In certain embodiments, the pharmaceutical composition is administered subcutaneously. In certain embodiments, the at least one additional therapeutic agent is IL-10.

In certain aspects, the application describes methods of treating or preventing an inflammatory-related condition, comprising administering an effective amount of the pharmaceutical composition described above. In certain embodiments, the inflammatory related condition is asthma. In certain embodiments, the inflammatory related condition is sepsis.

In certain aspects, the application describes methods of treating acute inflammation, comprising administering an effective amount of any of the above described pharmaceutical compositions.

In certain aspects, the application describes pharmaceutical compositions for the treatment of asthma comprising NVs derived from MSCs, wherein administration of the pharmaceutical composition results in reduced infiltration of T lymphocytes in the lungs of subjects with asthma compared to administration of a pharmaceutical composition comprising the same concentration of exosomes derived from MSCs.

In certain aspects, the application describes a method of preparing a pharmaceutical composition comprising a heterogeneous population of NVs for the prevention or treatment of inflammatory-related condition, the method comprising: providing a plurality of donor MSCs from a human subject; suspending the donor MSCs in a solution to create a MSC cell suspension; passing the suspension of MSCs through a plurality of membrane filters, thereby generating a heterogeneous population of NVs. In certain embodiments, the membrane filters comprise pores with diameters of 10 μm or less. In certain embodiments, the NVs have a diameter of between about 20 nm and 250 nm. In certain embodiments, the NVs have a substantial amount of nucleic acid within the NVs. In certain embodiments, the MSCs are human. In certain embodiments, the MSCs are derived from an autologous donor. In certain embodiments, the inflammatory related condition is selected from the group consisting of asthma, sepsis, infection, Rheumatoid arthritis, ulcerative colitis, Crohn's disease, tuberculosis, hepatitis, sinusitis, autoimmune disease, inflammatory bowel disease, pelvic inflammatory disease, ulcers, atherosclerosis, erythema, necrosis, vasculitis, ankylosing spondylitis, connective tissue disease, kidney disease, sarcoidosis, thyroiditis, osteoarthritis, Rheumatism, chronic inflammatory demyelinating polyneuropathy, pancreatitis, psoriatic arthritis, periodontitis, Behcet's disease, sinusitis, polymyalgia rheumatic, nephritis, diverticulitis, granulomatosis with polyangilitis, granuloma, encephalitis, immune-mediated inflammatory disease, esophagitis, gout, uveitis, myopathy, gallbladder disease, periodic fever syndrome, interstitial cystitis, peritonitis, appendicitis, Parkinson's disease, Alzheimer's, systemic lupus erythematous, fibromyalgia, diverticulitis, dermatitis and ankylosing spondylitis. In certain embodiments, the inflammatory related condition is asthma. In certain embodiments, the inflammatory related condition is sepsis.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 shows transmission electron microscopy images of NVs (left) and exosomes (right).

FIG. 2 is an electropherogram of RNA molecules isolated from NVs in comparison to RNA molecules isolated from exosomes.

FIG. 3 is an electropherogram of DNA molecules isolated from NVs in comparison to RNA molecules isolated from exosomes.

FIG. 4A shows the biodistribution of NVs labeled with Cy7 in mice 30 minutes after instillation (white arrow points to the lung). Control animals are shown in the left, animals that received the NVs intraperitoneally are displayed in the center, and animals that received intranasal instillation of NVs are displayed in the right.

FIG. 4B shows the biodistribution of NVs labeled with Cy7 in mice 6 hours after instillation in the lymph node (solid white arrow), the liver (larger dash arrow at center) and the lung (small dash arrow at top right). Control animals are shown in the left, animals that received the NVs intraperitoneally are displayed in the center, and animals that received intranasal instillation of NVs are displayed in the right.

FIG. 4C shows the biodistribution of NVs labeled with Cy7 in mice 24 hours after instillation in the lung (solid white arrow), the bladder (larger dash arrow at bottom left), and the pancreas (small dash arrow at center). Control animals are shown in the left, animals that received the NVs intraperitoneally are displayed in the center, and animals that received intranasal instillation of NVs are displayed in the right.

FIG. 5A is a graph showing total cellularity in BALF was decreased by both treatments. Bars represent mean±SD. One-way ANOVA followed by Dunnet's test was performed. *Significantly different from C-PBS ** Significantly different from OVA-PBS, p<0.05

FIG. 5B is a graph showing specific decrease in the number of eosinophils found in BALF of animals treated with exosomes or NVs. Bars represent mean±SD. One-way ANOVA followed by Dunnet's test was performed. *Significantly different from C-PBS ** Significantly different from OVA-PBS, p<0.05

FIG. 6 is a graph showing infiltration of T lymphocytes in the lung tissue was decreased by NV treatment. Measurements by flow cytometry. Bars represent mean±SD. One-way ANOVA followed by Dunnet's test was performed. *Significantly different from C-PBS ** Significantly different from OVA-PBS, p<0.05.

FIG. 7A is a graph showing Eotaxin-2 protein levels detected by ELISA in lung tissue lysates. Bars represent mean±SD. One-way ANOVA followed by Dunnet's test was performed. *Significantly different from C-PBS ** Significantly different from OVA-PBS, p<0.05.

FIG. 7B is a graph showing Eotaxin-2 protein levels detected by ELISA in BALF. Bars represent mean±SD. One-way ANOVA followed by Dunnet's test was performed. *Significantly different from C-PBS ** Significantly different from OVA-PBS, p<0.05.

FIG. 8A is a graph showing reduction in OMVs-induced pro-inflammatory cytokines (TNF-α) by NVs. OMVs (100 ng/mL) were added to the RAW 264.7 cells for 3 hours, followed by washing with PBS, and treatment of NVs. Supernatant concentrations of TNF-α and IL-6 15 hours later were measured by ELISA. *, P<0.05; **, P<0.01. Error bars indicate SEM. n=2.

FIG. 8B is a graph showing reduction in OMVs-induced pro-inflammatory cytokines (IL-6) by NVs. OMVs (100 ng/mL) were added to the RAW 264.7 cells for 3 hours, followed by washing with PBS, and treatment of NVs. Supernatant concentrations of TNF-α and IL-6 15 hours later were measured by ELISA. *, P<0.05; **, P<0.01. Error bars indicate SEM. n=2.

FIG. 9A is a diagram of the study protocol for investigation of inflammation in mice. E. coli OMVs (15 μg) were injected intraperitoneally once to provoke septic condition, followed by intraperitoneal injection of NVs (2×10⁹). Mice were then sacrificed at 6 h.

FIG. 9B shows images of eye exudates of mice at 6 h.

FIG. 9C is a graph of the body temperature of mice at 6 h. *, P<0.05; **, P<0.01; ***, P<0.001; one-way ANOVA with Tukey's multiple comparison test. Error bars indicate SEM. n=10

FIG. 9D is a graph of the body weight of mice at 6 h. *, P<0.05; **, P<0.01; ***, P<0.001; one-way ANOVA with Tukey's multiple comparison test. Error bars indicate SEM. n=10.

FIG. 10A is a graph of the percentage of neutrophils in the peritoneum of mice determined by FACS. *, P<0.05; **, P<0.01; ***, P<0.001; one-way ANOVA with Tukey's multiple comparison test. Error bars indicate SEM. n=10.

FIG. 10B is a graph of the percentage of monocytes in the peritoneum of mice determined by FACS. *, P<0.05; **, P<0.01; ***, P<0.001; one-way ANOVA with Tukey's multiple comparison test. Error bars indicate SEM. n=10.

FIG. 10C is a graph of inflammatory cytokine TNF-α in the peritoneum of mice. *, P<0.05; **, P<0.01; ***, P<0.001; one-way ANOVA with Tukey's multiple comparison test. Error bars indicate SEM. n=10.

FIG. 10D is a graph of inflammatory cytokine IL-6 in the peritoneum of mice. *, P<0.05; **, P<0.01; ***, P<0.001; one-way ANOVA with Tukey's multiple comparison test. Error bars indicate SEM. n=10.

FIG. 11A is a graph showing the number of total leukocytes in serum of mice. *, P<0.05; **, P<0.01; ***, P<0.001; one-way ANOVA with Tukey's multiple comparison test. Error bars indicate SEM. n=10.

FIG. 11B is a graph showing the number of platelets in serum of mice. *, P<0.05; ** P<0.01; ***, P<0.001; one-way ANOVA with Tukey's multiple comparison test. Error bars indicate SEM. n=10.

FIG. 11C is a graph showing inflammatory cytokine TNF-α in serum mice. *, P<0.05; ** P<0.01; ***, P<0.001; one-way ANOVA with Tukey's multiple comparison test. Error bars indicate SEM. n=10.

FIG. 11D is a graph showing inflammatory cytokine IL-6 in serum of mice. *, P<0.05; ** P<0.01; ***, P<0.001; one-way ANOVA with Tukey's multiple comparison test. Error bars indicate SEM. n=10.

FIG. 11E is a graph showing inflammatory cytokine KC in serum of mice. *, P<0.05; ** P<0.01; ***, P<0.001; one-way ANOVA with Tukey's multiple comparison test. Error bars indicate SEM. n=10.

FIG. 11F is a graph showing inflammatory cytokine RANTES in serum of mice. *, P<0.05; **, P<0.01; ***, P<0.001; one-way ANOVA with Tukey's multiple comparison test. Error bars indicate SEM. n=10.

FIG. 12A is a graph of the number of total leukocytes in BAL fluid. *, P<0.05; ** P<0.01; ***, P<0.001; one-way ANOVA with Tukey's multiple comparison test. Error bars indicate SEM. n=10.

FIG. 12B is a graph of the concentration of TNF-α in BAL fluid. *, P<0.05; **, P<0.01; *** P<0.001; one-way ANOVA with Tukey's multiple comparison test. Error bars indicate SEM. n=10.

FIG. 12C is a graph of the concentration of IL-6 in BAL fluid. *, P<0.05; **, P<0.01; *** P<0.001; one-way ANOVA with Tukey's multiple comparison test. Error bars indicate SEM. n=10.

FIG. 13A is a graph the concentration of IL-10 in conditioned media of RAW 264.7 cells pre-incubated with OMVs (100 ng/mL) for 3 h, and treated with various doses of NVs for 15 h (n=3).

FIG. 13B is a graph of the concentration of IL-10 in the peritoneal fluid at 6 h following OMVs administration (n=10).

FIG. 13C is a graph of the concentration of IL-10 in serum at 6 h following OMVs administration (n=10).

FIG. 13D is a graph showing serum concentrations of TNF-α of mice 6 h after i.p. injection with anti-IL-10 antibody (mIL-10) or isotype-matched control antibody together with OMVs, followed by injection NVs after 1 h. (n=5). *, P<0.05; **, P<0.01; ***, P<0.001; ns, not significant; one-way ANOVA with Tukey's multiple comparison test. Error bars indicate SEM.

FIG. 13E is a graph showing serum concentrations of IL-6 of mice 6 h after i.p. injection with anti-IL-10 antibody (mIL-10) or isotype-matched control antibody together with OMVs, followed by injection NVs after 1 h. (n=5). *, P<0.05; **, P<0.01; ***, P<0.001; ns, not significant; one-way ANOVA with Tukey's multiple comparison test. Error bars indicate SEM.

### DETAILED DESCRIPTION

### Advantages and Utility

Briefly, and as described in more detail below, described herein are methods for the production of NVs from MSCs useful for the treatment or prevention of inflammatory related conditions. The methods described herein also provide for a more efficient, less expensive method for the production of NVs in high concentrations. In certain embodiments, the methods described herein provide for the production of NVs from MSCs which are more effective than endogenous exosomes for use as an anti-inflammatory treatment.

### Definitions

Terms used in the claims and specification are defined as set forth below unless otherwise specified.

“Extracellular vesicles” or “EVs” refers to cell-derived vesicles comprising a membrane that encloses an internal space. EVs comprise all membrane-bound vesicles that have a smaller diameter than the cell from which they are derived. Generally, EVs range in diameter from 20 nm to 1000 nm, and may comprise various macromolecular cargo either within the internal space, displayed on the external surface of the EV, and/or spanning the membrane. Said cargo may comprise nucleic acids, proteins, carbohydrates, lipids, small molecules, and/or combinations thereof. By way of example and without limitation, EVs include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). More specifically, NVs and exosomes are types of EVs. EVs may be derived from a living or dead organism, explanted tissues or organs, and/or cultured cells.

“Nanovesicles” or “NVs” refers to a cell-derived small (between 20-250 nm in diameter, more preferably 30-150 nm in diameter) vesicles comprising a membrane that encloses an internal space, and which is generated from said cell by direct or indirect manipulation such that said nanovesicle or NV would not be produced by said producer cell without said manipulation. Appropriate manipulations of said producer cell include but are not limited to serial extrusion, treatment with alkaline solutions, sonication, or combinations thereof. The production of NVs may, in some instances, result in the destruction of said producer cell. Preferably, populations of NVs are substantially free of vesicles that are derived from producer cells by way of direct budding from the plasma membrane or fusion of the late endosome with the plasma membrane. The NV comprises lipid or fatty acid and polypeptide, and optionally comprises a payload (e.g. a therapeutic agent), a receiver (e.g. a targeting moiety), a polynucleotide (e.g. a nucleic acid, RNA, or DNA), a sugar (e.g. a simple sugar, polysaccharide, or glycan) or other molecules. The NV, once it is derived from a producer cell according to said manipulation, may be isolated from the producer cell based on its size, density, biochemical parameters, or a combination thereof.

As used herein, the term “exosome” refers to a cell-derived small (between 20-300 nm in diameter, more preferably 40-200 nm in diameter) vesicle comprising a membrane that encloses an internal space, and which is generated from said cell by direct plasma membrane budding or by fusion of the late endosome with the plasma membrane. Generally, production of exosomes does not result in the destruction of the producer cell. The exosome comprises lipid or fatty acid and polypeptide, and optionally comprises a payload (e.g. a therapeutic agent), a receiver (e.g. a targeting moiety), a polynucleotide (e.g. a nucleic acid, RNA, or DNA), a sugar (e.g. a simple sugar, polysaccharide, or glycan) or other molecules. The exosome can be derived from a producer cell, and isolated from the producer cell based on its size, density, biochemical parameters, or a combination thereof.

“Extrusion” refers to the serial passage of cells and/or cell fragments through filters comprising pores. The filters used for “extrusion” can be of any known material used in the art to filter cells or cell fragments. The pore sizes of the filters can be of any size useful for passing and/or separating cells and/or cell fragments. Cells and cell fragments may be exposed to other treatments or steps between passages of filters, such as washing, agitation, centrifugation or freezing.

“Inflammatory related condition”, “inflammatory related disease” or “inflammatory related disorder” refers to any condition or disease where increased inflammation is observed in a subject harboring the inflammatory related condition or disease. Inflammation may be contributing to the onset and/or the severity of the inflammatory related condition or disease. The inflammation may be a side-effect of the condition or disease or the inflammation may be a side-effect of another treatment for the condition, disorder or disease. Examples of inflammatory related conditions, disorders or diseases include, but are not limited to, asthma, sepsis, infection, Rheumatoid arthritis, ulcerative colitis, Crohn's disease, tuberculosis, hepatitis, sinusitis, autoimmune disease, inflammatory bowel disease, pelvic inflammatory disease, ulcers, atherosclerosis, erythema, necrosis, vasculitis, ankylosing spondylitis, connective tissue disease, kidney disease, sarcoidosis, thyroiditis, osteoarthritis, Rheumatism, chronic inflammatory demyelinating polyneuropathy, pancreatitis, psoriatic arthritis, periodontitis, Behcet's disease, sinusitis, polymyalgia rheumatic, nephritis, diverticulitis, granulomatosis with polyangilitis, granuloma, encephalitis, immune-mediated inflammatory disease, esophagitis, gout, uveitis, myopathy, gallbladder disease, periodic fever syndrome, interstitial cystitis, peritonitis, appendicitis, Parkinson's disease, Alzheimer's, systemic lupus erythematous, fibromyalgia, diverticulitis, dermatitis and ankylosing spondylitis. In some embodiments, the inflammatory related condition is an infection, for example a bacterial infection, a viral infection, or a parasitic infection.

“Administration,” “administering” and variants thereof means introducing a composition, such as a NV or an exosomes, or agent into a subject and includes concurrent and sequential introduction of a composition or agent. The introduction of a composition or agent into a subject is by any suitable route, including orally, pulmonarily, intranasally, parenterally (intravenously, intramuscularly, intraperitoneally, or subcutaneously), rectally, intralymphatically, or topically. Administration includes self-administration and the administration by another. A suitable route of administration allows the composition or the agent to perform its intended function. For example, if a suitable route is intravenous, the composition is administered by introducing the composition or agent into a vein of the subject. Administration can be carried out by any suitable route.

As used herein, the term “parent cell” includes any cell from which a NV may be isolated. The term also encompasses a cell that shares a protein, lipid, sugar, or nucleic acid component of the NV. For example, a “parent cell” includes cells which serve as a source for the NV membrane.

As used herein, the term “substantially” or “substantial” refers, e.g., to the presence, level, or concentration of an entity in a particular space, the effect of one entity on another entity, or the effect of a treatment. For example, an activity, level or concentration of an entity is substantially increased if the increase is 2-fold, 3-fold, 4-fold, 5-fold, 10-fold, 50-fold, 100-fold, or 1000-fold relative to a baseline. An activity, level or concentration of an entity is also substantially increased if the increase is 5%, 10%, 20%, 30%, 40%, 50%, 60%, 70%, 80%, 90%, 100%, 200%, or 500% relative to a baseline. An entity may be substantially present in a particular space if it can be detected by methods known in the art. An entity may not be substantially present in a particular space if it is present at levels below the limit of detection for assays and methods known in the art. In some embodiments, an entity may not be substantially present in a particular space if it is barely detectable but only in non-functional quantities or minute quantities that do not cause or change a phenotype. In other embodiments, an entity may not be substantially present in a particular population if it is present and can be detected only in a small number of constituents making up the population, e.g., less than 10%, 9%, 8%, 7%, 6%, 5%, 4%, 3% 2% or less than 1%, 0.5%, 0.1% of constituents of the population.

A “receiver,” as used herein, is an entity capable of interacting with a target, e.g., to associate with or bind to a target. A receiver can comprise or can consist essentially of a polypeptide. In some embodiments, the receiver comprises a polypeptide, a carbohydrate, a nucleic acid, a lipid, a polysaccharide (e.g., a glycan moiety), a small molecule, or a combination thereof. In embodiments in which the receiver is a naturally occurring compound or molecule, the receiver may be endogenous to the parent cell. In other embodiments in which a receiver is a naturally occurring compound or molecule, the receiver may be “synthetic” in the sense that it is an exogenous or heterologous compound or molecule with regard to its presence in the parent cell or NV. In other embodiments the receiver is “synthetic” in the sense that it is a man-made compound or molecule, such as a fusion or chimera, a non-naturally occurring polypeptide, carbohydrate, nucleic acid, lipid, or combination thereof, or a man-made small molecule or other therapeutic agent. For example, the receiver may comprise a fusion or chimera comprising one or more of an S domain, an A domain and a U domain. The S domain is a surface domain exposed to the environment around the NV, such as the circulatory system of a subject. The A domain is an anchor domain that attaches the S domain to the synthetic membrane of the NV. The U domain faces the unexposed side of or is located within the NV, i.e., the side that is not exposed to the external environment of the circulatory system of a subject. Irrespective of any domains, a receiver may be located on the surface of the NV or may be located within the complex. The receiver may be associated with the membrane of the NV, e.g., the receiver is anchored in, conjugated to or otherwise bound to the membrane. In some embodiments, the receiver may be conjugated to the membrane of the NV by chemical or enzymatic conjugation. In other embodiments, the receiver is not conjugated to the membrane. In some embodiments, the receiver is not associated with the membrane of the NV and is located within the membrane-encapsulated volume of the complex. In some embodiments, a receiver located within the NV does not substantially diffuse out of the complex and/or may not permeate the membrane. In other embodiments, the receiver may substantially diffuse out of the complex and/or may permeate the membrane. In some embodiments, the receiver is loaded, e.g., introduced into or put onto the NV. A receiver that is loaded is not biologically synthesized by the NV. A receiver suitable for loading may be, e.g., produced in a cell-based expression system, isolated from a biological sample, or chemically or enzymatically synthesized, and then loaded into or onto the NV. In some embodiments, the receiver may be further modified by the NV after loading. In other embodiments, the receiver is not modified after loading. In some embodiments, the receiver polypeptide is not loaded onto or into the complex. In some embodiments, the receiver is made, e.g., biologically synthesized by the NV. Typically a receiver polypeptide is expressed by the NV from an exogenous nucleic acid molecule (e.g., a DNA or mRNA) that was introduced into the complex. The receiver may bind to and/or sequester a target. Alternatively or in addition the receiver may exhibit a catalytic activity toward the target, e.g., the receiver may convert or modify the target, or may degrade the target. A product may then optionally be released from the receiver.

“Recipient cell” refers to a cell that interacts with, binds or associates with, receives a payload or uptakes a NV.

“Specifically binding” or “specifically interacting”, as used herein, describes any interaction between two entities (e.g., a target with a receiver, such as an antibody with an antigen, a receptor with a ligand, an enzyme with a substrate, biotin with avidin, etc.) that is saturable, often reversible and so competitive, as these terms are understood by those of ordinary skill in the chemical and biochemical arts. e.g., Specific binding involving biological molecules such as, e.g., proteins, peptides and nucleic acid occurs when one member of the binding pair has a site with a shape and distribution of charged, polar, or hydrophobic moieties such that the interaction of the cognate ligand with that site is characterized by favorable energetics (i.e., a negative free energy of binding). The specificity of the interaction may be measured or expressed as a binding constant (Kd). The Kd may range from a mM range to a pM range, including M ranges and nM ranges. Typical Kd values are below about 10⁻⁶M, below about 10⁻⁷ M, below about 10⁻⁸ M, and in some embodiments below about 10⁻⁹ M.

A “target,” as used herein, is an entity capable of interacting with a NV and/or a receiver, e.g., to associate with or bind to a receiver. A “target” includes, but is not limited to a polypeptide (e.g., an antibody or antibody-related polypeptide, a complement constituent, an amyloid protein, a pathogen, a toxin, a prion), a molecule (e.g., a metabolite, a steroid, a hormone, a carbohydrate; an oligosaccharide; a chemical; a polysaccharide, a DNA; an RNA; a lipid, an amino acid, an element, a toxin or pathogen), a complex (e.g., an immune complex), or a cell (e.g., a cancer cell, a macrophage, a bacterium, a fungus, a virus, or a parasite). A target is intended to be detected, diagnosed, impaired, destroyed or altered (e.g., functionally complemented) by the methods provided herein. The specific target may occur free or is associated with other entities in the circulatory system of a subject.

“In vivo” refers to processes that occur in a living organism.

“Mammal” as used herein includes both humans and non-humans and include but is not limited to humans, non-human primates, canines, felines, murines, bovines, equines, and porcines.

“Sufficient amount” means an amount sufficient to produce a desired effect, e.g., an amount sufficient to modulate inflammation in a cell.

“Therapeutically effective amount” is an amount that is effective to ameliorate a symptom of a disease (i.e., an inflammatory-related condition). A therapeutically effective amount can be a “prophylactically effective amount” as prophylaxis can be considered therapy. The term “therapeutically effective amount” or “effective amount” is an amount of an agent being administered to a subject sufficient to effect beneficial or desired clinical results, pharmacologic and/or physiologic effects. An effective amount can be administered in one or more administrations. An effective amount is typically sufficient to palliate, ameliorate, stabilize, reverse, slow or delay the progression of the disease state. The effective amount thus refers to a quantity of an agent or frequency of administration of a specific quantity of an agent sufficient to reasonably achieve a desired therapeutic and/or prophylactic effect. For example, it may include an amount that results in the prevention of, treatment of, or a decrease in, the symptoms associated with a disease or condition that is being treated, e.g., the diseases or medical conditions associated with a target polypeptide. The amount of a therapeutic composition administered to the subject will depend on the type and severity of the disease and on the characteristics of the individual, such as general health, pathologic conditions, diets, age, sex, body weight and tolerance to drugs. It will also depend on the degree, severity and type of disease. Further, the effective amount will depend on the methods of formulation and administration used, e.g., administration time, administration route, excretion speed, and reaction sensitivity. The skilled artisan will be able to determine appropriate dosages depending on these and other factors. The compositions can also be administered in combination with one or more additional therapeutic compounds. A desirable dosage of the pharmaceutical composition may be in the range of about 0.001 to 100 mg/kg for an adult. In one example, an intravenous administration is initiated at a dose which is minimally effective, and the dose is increased over a pre-selected time course until a positive effect is observed. Subsequently, incremental increases in dosage are made limiting to levels that produce a corresponding increase in effect while taking into account any adverse effects that may appear. Non-limited examples of suitable dosages can range, for example, from 1×10⁷ to 1×10¹⁰, 1×10¹⁰ to 1×10¹⁴, from 1×10¹¹ to 1×10¹³, or from 5×10¹¹ to 5×10¹² NVs of the present invention. Specific examples include about 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², or more NVs of the present invention. Each dose of NVs can be administered at intervals such as once daily, once weekly, twice weekly, once monthly, or twice monthly.

Abbreviations used in this application include the following: “NV” refers to nanovesicles; “EV” revers to extracellular vesicles.

It must be noted that, as used in the specification and the appended claims, the singular forms “a,” “an” and “the” include plural referents unless the context clearly dictates otherwise.

### METHODS OF THE INVENTION

Disclosed herein are methods for the production of NVs that possess similar properties to those of exosomes in terms of size, membrane composition and biological activity, and can be artificially produced through the extrusion of cells.

Preparation of NVs

NVs are prepared from MSCs. In certain embodiments the MSCs are primary bone marrow-derived MSCs. Primary MSCs can be harvested from subject and identified and/or isolated by any method known in the art. MSCs can be isolated and/or enriched by detection of markers known to be expressed on MSCs by methods such as flow cytometry. In certain embodiments, the MSCs are derived from a subject (i.e., an autologous donor) that is later administered the NVs that are obtained from the MSCs. In certain embodiments, the MSCs are derived from a subject (i.e., a heterologous donor) and the NVs that are produced from the MSCs are administered to a different recipient. In certain embodiments, isolated MSCs may be cultured and expanded ex vivo to provide a variety of source material to generate NVs. In certain embodiments, the MSCs have been passaged in cell culture. In certain embodiments, primary MSCs have been passaged between 3 and 5 times prior to preparation of NVs. In certain embodiments, the MSCs have not been passaged in cell culture. In certain embodiments, the MSCs have been frozen prior to use. In certain embodiments, the MSCs have not been frozen prior to use.

In certain embodiments, MSCs have been modified to express or not to express genes of interest prior to production of NVs. In certain embodiments, MSCs are characterized and/or sorted (e.g., by flow cytometry) for expression or lack of expression of markers or genes of interest.

In certain embodiments, the MSCs are cultivated in cell culture medium. In certain embodiments, the MSCs are cultivated in cell culture medium lacking serum. In certain embodiments, the MSCs are cultivated in alpha MEM medium. In certain embodiments, the MSC's are cultivated in alpha MEM medium supplemented with serum. In certain embodiments, the MSC's are cultivated in alpha MEM medium supplemented with serum. 15% fetal bovine serum and 1% antibiotic solution (100 units/ml penicillin and 100 mg/ml streptomycin)

In certain embodiments, MSCs are incubated in serum free medium prior to production of NVs extrusion. In certain embodiments, MSCs are incubated for any time between 1 second and 1 week in serum free medium prior to production of NVs. In certain embodiments, MSCs are incubated for 72 h, 48 h, 36 h, 24 h, 12 h, 6 h, 3 h, 2 h, 1 h, 30 min, 15 min, 10 min, 5 min, 1 min, 30 s or is prior to extrusion of the MSCs. In certain embodiments, after 24 h of incubation of MSCs in serum-free medium, the cells were washed with phosphate buffered saline (PBS, pH 7.4) and detached from the cell culture flasks or plates.

In certain embodiments, to prepare the NVs, MSC cell suspensions are passed through one or more filters having a pore size 100 μm or smaller. In certain embodiments, MSC cell suspensions are passed through one or more polycarbonate membrane filters having a pore size 100 μm or smaller. In certain embodiments, MSC cell suspensions are passed through one or more filters having a pore size 50 μm or smaller, 40 μm or smaller, 30 μm or smaller, 20 μm or smaller, 10 μm or smaller, 5 μm or smaller or 1 μm or smaller.

In certain embodiments, to prepare the NVs, MSC cell suspensions are passed through one or more filters between 1 and 100 times. In certain embodiments, MSC cell suspensions are passed through one or more filters less than 50 times, less than 40 times, less than 30 times, less than 20 times, or less than 10 times. In certain embodiments, MSC cell suspensions are passed through one or more filters 20 times, 19 times, 18 times, 17 times, 16 times, 15 times, 14 times, 13 times, 12 times, 11 times, 10 times, 9 times, 8 times, 7 times, 6 times, 5 times, 4 times, 3 times, 2 times or 1 time.

In certain embodiments, to prepare the NVs, MSC cell suspensions are passed between 1 and 50 times through each of the polycarbonate membrane filters with a pore size of 10 μm, 5 μm and 1 μm, in that order. In certain embodiments, MSC cell suspensions are passed five times through each of the polycarbonate membrane filters with a pore size of 10 μm, 5 μm and 1 μm, in that order.

In certain embodiments, to prepare the NVs, MSC cell suspensions are passed through filters using an extruder or mini-extruder. In certain embodiments, MSC cell suspensions are passed through filters using a syringe. In certain embodiments, MSC cell suspensions are passed through filters using a vacuum apparatus.

In certain embodiments, to prepare the NVs, MSC cell suspension effluent from the filter passages are ultracentrifuged to further separate the MVs from the other cellular material in the effluent. In certain embodiments, the cell effluent is mixed with one or more solutions comprising an alcohol prior to the ultracentrifugation step. In certain embodiments, the cell effluent is mixed with one or more solutions comprising iodixanol prior to the ultracentrifugation step. In certain embodiments, the cell effluent is mixed with two solutions comprising 50% and 10% iodixanol prior to the ultracentrifugation step.

In certain embodiments, the cell effluent is ultracentrifuged at a force between 20,000 and 200,000×g for between 1 min and 24 h. In certain embodiments, the cell effluent is ultracentrifuged at a force between 50,000 and 150,000×g for between 1 min and 24 h. In certain embodiments, the cell effluent is ultracentrifuged at a force between 75,000 and 150,000×g for between 1 min and 24 h. In certain embodiments, the cell effluent is ultracentrifuged at a force between 100,000 and 120,000×g for between 1 min and 24 h.

In certain embodiments, the cell effluent is ultracentrifuged at a force between 75,000 and 150,000×g for between 1 min and 24 h, between 15 min and 24 h, between 30 min and 12 h, between 1 h and 12 h, between 1 h and 6 h, between 1 h and 3 h, between 1 h and 2 h, between 30 min and 3 h. In certain embodiments, the cell effluent is ultracentrifuged at a force of 100,000×g for between 1 h and 3 h. In certain embodiments, the cell effluent is ultracentrifuged at a force of 100,000×g for 2 h. In certain embodiments, the layers formed between 50% iodixanol and 10% iodixanol after ultracentrifugation at 100,000×g for 2 hours was collected and considered NVs.

In certain embodiments, NVs are frozen after extrusion and/or after ultracentrifugation.

In certain embodiments, NVs are further processed, modified, purified or characterized after extrusion and/or ultracentrifugation prior to administration or formulation into a pharmaceutical composition for administration to a patient in need thereof.

Loading of NV's

In certain embodiments, NVs are loaded with one or more therapeutic payloads. Suitable payloads include, without limitation, pharmacologically active drugs and genetically active molecules, including additional anti-inflammatory agents, hormones or hormone antagonists, ion channel modifiers, antineoplastic agents, and neuroactive agents.

NVs may optionally be loaded with payloads such as peptides, proteins, DNA, RNA, siRNA, and other macromolecules and small therapeutic molecules. In some embodiments, the payload is transferred to a cell, e.g., a parent erythroid cell or parent platelet by applying controlled injury to the cell for a predetermined amount of time in order to cause perturbations in the cell membrane such that the payload can be delivered to the inside of the cell (e.g., cytoplasm). In some embodiments the payload is transferred to a NV isolated from a parent cell, e.g., a parent erythroid cell or parent platelet, by applying controlled injury to the NV for a predetermined amount of time in order to cause perturbations in the cell membrane such that the payload can be delivered to the inside of the NV. In other embodiments, the payload is transferred to a NV isolated from a parent cell, e.g., by applying controlled injury to an isolated membrane derived from a parent cell for a predetermined amount of time in order to cause perturbations in the cell membrane such that the payload can be delivered to the inside of the isolated membrane.

In some embodiments the payload of the NV may be loaded within the membrane or interior portion of the NV.

The payload may be a therapeutic agent selected from a variety of known small molecule pharmaceuticals. Alternatively, the payload may be may be a therapeutic agent selelcted from a variety of macromolecules, such as, e.g., an inactivating peptide nuclei acid (PNA), an RNA or DNA oligonucleotide aptamer, an interfering RNA (iRNA), a peptide, or a protein.

In some embodiments, the payload of the NV is a nucleic acid molecule, e.g. mRNA or DNA, and the NV targets the payload to the cytoplasm of the recipient cell, such that the nucleic acid molecule can be translated (if mRNA) or transcribed and translated (if DNA) and thus produce the polypeptide encoded by the payload nucleic acid molecule within the recipient cell. In an embodiment, the polypeptide encoded by the payload nucleic acid molecule is secreted by the target cell, thus modulating the systemic concentration or amount of the polypeptide encoded by the payload nucleic acid molecule in the subject. In an embodiment, the polypeptide encoded by the payload nucleic acid molecule is not secreted by the recipient cell, thus modulating the intracellular concentration or amount of the polypeptide encoded by the payload nucleic acid molecule in the subject. In an embodiment, the polypeptide encoded by the payload nucleic acid molecule increases the anti-inflammatory effect of the NV on the target cell or to other cells or tissues in the subject. In an embodiment, the polypeptide encoded by the payload nucleic acid molecule does not promote anti-inflammatory effects on the recipient cell or other cell or tissue in the subject, but is therapeutically beneficial or corrects a disease phenotype.

In some embodiments, the payload of the NV is a miRNA or pre-miRNA molecule, and the NV targets the payload to the cytoplasm of the recipient cell, such that the miRNA molecule can silence a native mRNA in the recipient cell.

In some embodiments, the payload may be engineered for specific trafficking from the parent cell into the NV. In some embodiments, the receiver or payload may be directed for trafficking by an addition of a molecule to the payload (e.g. conjugation or fusion of another molecule). In certain embodiments, the additional molecule may be appended via a linker. In other embodiments, the payload may be directed for trafficking by modifying the payload composition (e.g., a nucleotide change for nucleic acid payloads or amino acid change for polypeptide payloads).

In some embodiments, a nucleic acid payload may be engineered for specific trafficking from the parent cell into the NV. In certain embodiments, a nucleic acid payload (e.g., mRNA or miRNA) may comprise a sequence in the coding or noncoding region that targets the nucleic acid to the NV. In certain embodiments, the noncoding region may include a 3′ UTR or 5′ UTR.

In some embodiments, the payload of the NV may be a membrane protein delivered to the plasma membrane or endosomal membrane of the recipient cell.

In some embodiments, the payload is a therapeutic agent, such as a small molecule drug or a large molecule biologic. Large molecule biologics include, but are not limited to, a protein, polypeptide, or peptide, including, but not limited to, a structural protein, an enzyme, a cytokine (such as an interferon and/or an interleukin), a polyclonal or monoclonal antibody, or an effective part thereof, such as an Fv fragment, which antibody or part thereof, may be natural, synthetic or humanized, a peptide hormone, a receptor, or a signaling molecule.

Large molecule biologics are immunoglobulins, antibodies, Fv fragments, etc., that are capable of binding to antigens in an intracellular environment. These types of molecules are known as “intrabodies” or “intracellular antibodies.” An “intracellular antibody” or an “intrabody” includes an antibody that is capable of binding to its target or cognate antigen within the environment of a cell, or in an environment that mimics an environment within the cell. Selection methods for directly identifying such “intrabodies” include the use of an in vivo two-hybrid system for selecting antibodies with the ability to bind to antigens inside mammalian cells. Such methods are described in PCT/GB00/00876, incorporated herein by reference. Techniques for producing intracellular antibodies, such as anti-β-galactosidase scFvs, have also been described in Martineau et al., J Mol Biol 280:117-127 (1998) and Visintin et al., Proc. Natl. Acad. Sci. USA 96:11723-1728 (1999).

Large molecule biologics include but are not limited to, at least one of a protein, a polypeptide, a peptide, a nucleic acid, a virus, a virus-like particle, an amino acid, an amino acid analogue, a modified amino acid, a modified amino acid analogue, a steroid, a proteoglycan, a lipid and a carbohydrate or a combination thereof (e.g., chromosomal material comprising both protein and DNA components or a pair or set of effectors, wherein one or more convert another to active form, for example catalytically).

A large molecule biologic may include a nucleic acid, including, but not limited to, an oligonucleotide or modified oligonucleotide, an antisense oligonucleotide or modified antisense oligonucleotide, an aptamer, a cDNA, genomic DNA, an artificial or natural chromosome (e.g., a yeast artificial chromosome) or a part thereof, RNA, including an siRNA, a shRNA, mRNA, tRNA, rRNA or a ribozyme, or a peptide nucleic acid (PNA); a virus or virus-like particles; a nucleotide or ribonucleotide or synthetic analogue thereof, which may be modified or unmodified.

The large molecule biologic can also be an amino acid or analogue thereof, which may be modified or unmodified or a non-peptide (e.g., steroid) hormone; a proteoglycan; a lipid; or a carbohydrate. If the large molecule biologic is a polypeptide, it can be loaded directly into, e.g., a parent erythroid cell or a parent platelet according to the methods described herein. Alternatively, an exogenous nucleic acid encoding a polypeptide, which sequence is operatively linked to transcriptional and translational regulatory elements active in a parent cell at a target site, may be loaded.

Small molecules, including inorganic and organic chemicals, may also be used as payloads of the NVs described herein.

Specific examples of therapeutic payloads of certain embodiments of the invention include: inhibitors of Myeloid Differentiation Factor 88 (Myd88) and GATA-3. Myd88 is a general adaptor protein that plays an important role in the Toll-like receptor (TLR) family signaling (Biol Res. 2007; 40(2):97-112). Pharmacologic inhibition or silencing RNA of Myd88 have been extensively in inflammatory diseases such as sepsis and acute myocardial infarction (J Clin Cell Immunol. 2011; 2(5): 1000e102) and (J Cardiovasc Pharmacol. 2010; 55(4):385-90).

Examples of inhibitors of myd88 include, pharmacologic inhibition, silencing RNA (siRNA) targeted against Myd88 or synthetic peptides (e.g., ST2825, NBP2-29328), which mimic Toll/interlukin-1 receptor (TIR) domain of Myd88. GATA-3 is a transcription factor involved in allergic inflammation, through the transcriptional modulation of cytokines production and Th2 responses especially in TCD4 cells (Cell. 1997; 89, 587-596) and (J Immunol 2016; 196:4423-4425). Knockdown and inhibition of GATA-3 has been shown to decrease the inflammation and airway hyperresponsiveness in a murine model of asthma (Mol Ther 2008; 16 (1): 60-65).

NVs may comprise two or more payloads, including mixtures, fusions, combinations and conjugates, of atoms, molecules, etc. as disclosed herein, for example including but not limited to, a nucleic acid combined with a polypeptide; two or more polypeptides conjugated to each other; a protein conjugated to a biologically active molecule (which may be a small molecule such as a prodrug); and the like.

In some embodiments, the application describes pharmaceutical compositions comprising one or more therapeutic agents and the NVs described herein. In some embodiments, the NVs are co-administered with of one or more separate therapeutic agents, wherein co-administration includes administration of the separate therapeutic agent before, after or concurrent with administration of the NV.

The NVs may also be labeled with one or more positive markers that can be used to monitor over time the number or concentration of NVs in the blood circulation of an individual. The overall number of NVs will decay over time following initial transfusion. In some embodiments, the signal from one or more positive markers are correlated with that of an activated molecular marker, generating a proportionality of signal that is independent of the number of NVs remaining in the circulation. Suitable fluorescent compounds include those that are approved by the Food & Drug Administration for human use including but not limited to fluorescein, indocyanin green, and rhodamine B. For example, parent cells or NVs may be non-specifically labeled with fluorescein isothiocyanate (FITC; Bratosin et al., Cytometry 46:351-356 (2001)). For example, a solution of FITC-labeled lectins in phosphate buffered saline (PBS) with 0.2 mM phenylmethysulfonyl fluoride (PMSF) is added to an equal volume of isolated parent erythroid cells or parent platelets or NVs in the same buffer. The cells are incubated with the FITC-labeled lectins for 1 h at 4° C. in the dark. The lectins bind to sialic acids and beta-galactosyl residues on the surface of the parent erythroid cells or NVs.

Other dyes may be useful for tracking NVs in human and non-human circulation. A number of reagents may be used to non-specifically label a NV. For example, parent erythroid cells or parent platelets or NVs may be labeled with PKH26 Red (See, e.g., Bratosin, et al., (1997) Cytometry 30:269-274). Parent erythroid cells or parent platelets (1-3×10⁷ cells) are suspended in 1 ml of diluent and rapidly added to 1 ml or 2 μM PKH26 dissolved in the same diluent. The mixture is mixed by gentle pipetting and incubated at 25° C. for 2-5 min with constant stirring. The labeling may be stopped by adding an equal volume of human serum or compatible protein solution (e.g., 1% bovine serum albumin). After an additional minute, an equal volume of cell culture medium is added and the cells are isolated by centrifugation at 2000×g for 5 min. Cells are washed three times by repeated suspension in cell culture medium and centrifugation. PHK26-labeled NVs may be monitored with a maximum excitation wavelength of 551 nm and a maximum emission wavelength of 567 nm.

NVs may be tracked in vivo using VivoTag 680 (VT680; VisEn Medical, Woburn, Mass., USA), a near-infrared fluorochrome with a peak excitation wavelength of 670±5 nm and a peak emission wavelength of 688±5 nm. VT680 also contains an amine reactive NHS ester which enables it to cross-link with proteins and peptides. The surface of parent cells, e.g., parent erythroid cells or parent platelets or of NVs may be labeled with VT680 (See, e.g., Swirski, et al., (2007) PloS ONE 10:e1075). For example, 4×10⁶ cells/ml are incubated with VT680 diluted in complete culture medium at a final concentration of 0.3 to 300 jag/ml for 30 min at 37° C. The cells are washed twice with complete culture medium after labeling. Cells may be non-specifically labeled based on proteins expressed on the surface of the parent cell or the NV. Alternatively, a specific protein, such as a receiver may be labeled with VT680. In some embodiments, a protein or peptide may be directly labeled with VT680 ex vivo and subsequently either attached to the surface of the cell or incorporated into the interior of the cell using methods described herein. In vivo monitoring may, for example, be performed using the dorsal skin fold. Laser scanning microscopy may be performed using, for example, an Olympus IV 100 in which VT680 is excited with a red laser diode of 637 nm and detected with a 660/LP filter. Alternatively, multiphoton microscopy may be performed using, for example, a BioRad Radiance 2100 MP centered around an Olympus BX51 equipped with a 20×/0.95 NA objective lens and a pulsed Ti:Sapphire laser tuned to 820 nm. The latter wavelength is chosen because VT680 has a peak in its two-photon cross-section at 820 nm.

Alternatively or in addition, a NV may be labeled with other red and/or near-infrared dyes including, for example, cyanine dyes such as Cy5, Cy5.5, and Cy7 (Amersham Biosciences, Piscataway, N.J., USA) and/or a variety of Alexa Fluor dyes including Alexa Fluor 633, Alexa Fluor 635, Alexa Fluor 647, Alexa Fluor 660, Alexa Fluor 680, Alexa Fluor 700 and Alexa Fluor 750 (Molecular Probes-Invitrogen, Carlsbad, Calif., USA). Additional suitable fluorophores include IRD41 and IRD700 (LI-COR, Lincoln, Nebr., USA), NIR-1 and 1C5-OSu (Dejindo, Kumamotot, Japan), LaJolla Blue (Diatron, Miami, Fla., USA), FAR-Blue, FAR-Green One, and FAR-Green Two (Innosense, Giacosa, Italy), ADS 790-NS and ADS 821-NS (American Dye Source, Montreal, Calif.). Quantum dots (Qdots) of various emission/excitation properties may also be used for labeling NVs (See, e.g., Jaiswal et al., Nature Biotech. 21:47-51 (2003)). Many of these fluorophores are available from commercial sources either attached to primary or secondary antibodies or as amine-reactive succinimidyl or monosuccinimidyl esters, for example, ready for conjugation to a protein or proteins either on the surface or inside the NV.

Magnetic nanoparticles may be used to track NVs in vivo using high resolution MRI (Montet-Abou et al., Molecular Imaging 4:165-171 (2005)). Magnetic particles may be internalized by several mechanisms. Magnetic particles may be taken up by a parent cell, e.g., a parent erythroid cell or a parent platelet or by a NV through fluid-phase pinocytosis or phagocytosis. Alternatively, the magnetic particles may be modified to contain a surface agent such as, for example, a membrane translocating HIV TAT peptide which promotes internalization. In some instances, a magnetic nanoparticle such as, for example, Feridex IV®, an FDA approved magnetic resonance contrast reagent, may be internalized into, e.g., parent MSCs or NVs in conjunction with a transfection agent such as, for example, protamine sulfate (PRO), polylysine (PLL), and lipofectamine (LFA).

Description of NVs

In certain embodiments, the NVs are a heterogeneous population of membrane bound vesicles.

In some embodiments, the NVs are assessed for their basic physical properties, e.g., size, mass, volume, diameter, buoyancy, density, and membrane properties, e.g., viscosity, deformability fluctuation, and fluidity. In some embodiments, the NVs are assessed for the physiological properties, e.g., biodistribution profile, cellular uptake, half-life, pharmacodynamics, potency, dosing, immune response, loading efficiency, stability, or reactivity to other compounds.

In an embodiment, the diameter of the NVs is measured by microscopy or by automated instrumentation, e.g., a hematological analysis instrument. In certain embodiments, the diameter of the NVs is between about 20-1,000 nanometers. In some embodiments, the NV has a longest dimension between about 20-1000 nm, such as between about 20-100 nm, 20-200 nm, 20-300 nm, 20-400 nm, 20-500 nm, 20-600 nm, 20-700 nm, 20-800 nm, 20-900 nm, 30-100 nm, 30-200 nm, 30-300 nm, 30-400 nm, 30-500 nm, 30-600 nm, 30-700 nm, 30-800 nm, 30-900 nm, 40-100 nm, 40-200 nm, 40-300 nm, 40-400 nm, 40-500 nm, 40-600 nm, 40-700 nm, 40-800 nm, 40-900 nm, 50-150 nm, 50-500 nm, 50-750 nm, 100-200 nm, 100-500 nm, or 500-1000 nm.

In certain embodiments, the NVs are heterogeneous in size, mass, volume, diameter, buoyancy, density, and membrane properties, e.g., viscosity, deformability fluctuation, and fluidity.

In some embodiments, the NVs comprise a substantial amount of nucleic acid within the NVs. In certain embodiments, the NVs comprise a substantial amount of RNA within the NVs. In certain embodiments, the NVs comprise a detectable amount of DNA. In certain embodiments, the amount of nucleic acid within the NVs is substantially greater than the amount of nucleic acid found within naturally occurring exosomes from the same type of cells.

In certain embodiments, NVs comprises a membrane that sediments at approximately 1,000-200,000 g and comprises a density of approximately 0.8-1.4 g/ml. In some embodiments, the membrane comprises phosphatidylcholine, sphingomyelin, lysophosphatidylcholine, phosphatidylethanolamine, phosphatidylserine, phosphatidylinositol, phosphatidic acid or cholesterol. In some embodiments, the membrane comprises plasma membrane from MSCs. In some embodiments, the membrane comprises membranes from MSCs other than the plasma membrane.

In an embodiment, approximately 52% of the membrane mass is protein, approximately 40% is lipid, and approximately 8% is carbohydrate. In one embodiment, approximately 7% of the carbohydrate content is comprised of glycosphingolipids and approximately 93% of the carbohydrate content is comprised of O-linked and N-linked oligosaccharides on membrane-associated polypeptides.

In one embodiment the mass ratio of lipid to protein in the NV is less than 1:1000, approximately 1:1000, approximately 1:500, approximately 1:250, approximately 1:100, approximately 1:50, approximately 1:25, approximately 1:10, approximately 1:9, approximately 1:8, approximately 1:7, approximately 1:6, approximately 1:5, approximately 1:4, approximately 1:3, approximately 1:2, approximately 1:1, approximately 2:1, approximately 3:1, approximately 4:1, approximately 5:1, approximately 6:1, approximately 7:1, approximately 8:1, approximately 9:1, approximately 10:1, approximately 25:1, approximately 50:1, approximately 100:1, approximately 250:1, approximately 500:1, approximately 1000:1, or greater than approximately 1000:1.

In an embodiment the mass ratio of lipid to carbohydrate in the NV is less than 1:1000, approximately 1:1000, approximately 1:500, approximately 1:250, approximately 1:100, approximately 1:50, approximately 1:25, approximately 1:10, approximately 1:9, approximately 1:8, approximately 1:7, approximately 1:6, approximately 1:5, approximately 1:4, approximately 1:3, approximately 1:2, approximately 1:1, approximately 2:1, approximately 3:1, approximately 4:1, approximately 5:1, approximately 6:1, approximately 7:1, approximately 8:1, approximately 9:1, approximately 10:1, approximately 25:1, approximately 50:1, approximately 100:1, approximately 250:1, approximately 500:1, approximately 1000:1, or greater than approximately 1000:1.

In an embodiment the mass ratio of carbohydrate to protein in the NV is less than 1:1000, approximately 1:1000, approximately 1:500, approximately 1:250, approximately 1:100, approximately 1:50, approximately 1:25, approximately 1:10, approximately 1:9, approximately 1:8, approximately 1:7, approximately 1:6, approximately 1:5, approximately 1:4, approximately 1:3, approximately 1:2, approximately 1:1, approximately 2:1, approximately 3:1, approximately 4:1, approximately 5:1, approximately 6:1, approximately 7:1, approximately 8:1, approximately 9:1, approximately 10:1, approximately 25:1, approximately 50:1, approximately 100:1, approximately 250:1, approximately 500:1, approximately 1000:1, or greater than approximately 1000:1.

In an embodiment the area occupancy of protein in the NV is approximately 23% and the area occupancy of lipid in the NV is approximately 77%.

In certain embodiments, the NVs comprise nucleic acid. The term “nucleic acid,” in its broadest sense, includes any compound and/or substance that is or can be incorporated into an oligonucleotide chain. Exemplary nucleic acids for include, but are not limited to, one or more of DNA, RNA, hybrids thereof, RNAi-inducing agents, RNAi agents, siRNAs, shRNAs, miRNAs, antisense RNAs, ribozymes, catalytic DNA, RNAs that induce triple helix formation, aptamers, vectors, etc. In certain embodiments, the NVs comprise RNA. In certain embodiments, the NVs comprise DNA. In certain embodiments, the NVs comprise both DNA and RNA or any combination of nucleic acids described above.

Anti-Inflammatory Activity of NVs

In certain embodiments, administration of compositions comprising NVs to subjects suspected of having or developing an immune-related condition, disorder or disease results in reduced inflammation in the subjects. In certain embodiments, administration of NVs to subjects results in the reduction of the number of immune cells in tissues of subjects suspected of having increased inflammation. In certain embodiments, reduced numbers of T-lymphocytes are observed in tissues of subjects suspected of having increased inflammation compared to untreated subjects. In certain embodiments, reduced numbers of eosinophils are observed in tissues of subjects suspected of having increased inflammation compared to untreated subjects. In certain embodiments, reduced numbers of macrophages are observed in tissues of subjects suspected of having increased inflammation compared to untreated subjects. In certain embodiments, reduced amount of immune cell derived cytokines are observed in tissues of subjects suspected of having increased inflammation compared to untreated subjects.

Receivers

Provided herein are receivers that are exhibited by NVs. In some embodiments, a receiver is capable of interacting with a target, e.g., to associate with or bind to a target. A receiver can comprise or may consist essentially of a polypeptide. In some embodiments, the receiver comprises a polypeptide, a carbohydrate, a nucleic acid, a lipid, a small molecule, or a combination thereof. In some embodiments receivers do not interact with a target but act as payloads to be delivered by the NV to a cell, tissue or other site in the body of a subject.

In some embodiments, the interaction of the receiver with a target comprises binding, degrading, cleaving, sequestering, regulating and/or signaling the target.

In other embodiments, the interaction of the receiver with a target comprises altering an activity of the target. In other embodiments, the interaction of the receiver with a target comprises altering the composition of the target. In other embodiments, the interaction of the complex with a target comprises reducing an activity of the target. In other embodiments, the interaction of the complex with a target comprises inactivating the target.

In some embodiments, receivers comprise polypeptides. Receiver polypeptides may range in size from 6 amino acids to 3000 amino acids and may exceed 6, 10, 15, 20, 25, 30, 35, 40, 50, 60, 70, 80, 90, 100, 150, 200, 300, 400 or may exceed 500 amino acids. Receiver polypeptides may range in size from about 20 amino acids to about 500 amino acids, from about 30 amino acids to about 500 amino acids or from about 40 amino acids to about 500 amino acids.

In some embodiments, the receiver polypeptide comprises a chimeric or fusion protein which may comprise two or more distinct protein domains. These chimeric receivers are heterologous or exogenous in the sense that the various domains are derived from different sources, and as such, are not found together in nature and can be encoded e.g., by exogenous nucleic acids. Receiver polypeptides can be produced by a number of methods, many of which are well known in the art and also described herein. For example, receiver polypeptides can be obtained by extraction (e.g., from isolated cells), by expression of an exogenous nucleic acid encoding the receiver polypeptide, or by chemical synthesis. Receiver polypeptides can be produced by, for example, recombinant technology, and expression vectors encoding the polypeptide introduced into host cells (e.g., by transformation or transfection) for expression of the encoded receiver polypeptide.

In some embodiments, the receiver polypeptide is associated with the membrane of the NV. In other embodiments, the receiver polypeptide is not associated with the membrane of the NV.

In certain embodiments, the polypeptide receiver is located on the surface and is exposed to the environment around the NV. In some embodiments, the polypeptide receiver is located inside and faces the unexposed side of the NV.

In some embodiments, the receiver interacts with a circulating pathogen, such as a virus or a bacterium. In some embodiments, the functional parent erythroid cell expresses an exogenous gene encoding an antibody, scFv, or nanobody specific for the circulating pathogen. The antibody, scFv, or nanobody may be expressed as a fusion protein. In other embodiments, the antibody, scFv, or nanobody receiver or another receiver with affinity to circulating pathogens is loaded into or onto the parent erythroid cell. The antibody, scFv, or nanobody receiver or the other receiver with affinity to circulating pathogens may be localized intracellularly or extracellularly. In some embodiments, the receiver is specific for a viral or bacterial antigen, such as a surface, envelope or capsid antigen.

In an embodiment, the receiver comprises a polypeptide that comprises an amino acid sequence derived from G protein-linked transmembrane receptors. In one embodiment, the functional parent erythroid cell expresses an exogenous gene encoding an amino acid sequence derived from G protein-linked transmembrane receptors. The G protein-linked transmembrane receptors receiver may be expressed as a full-length protein or a fragment thereof. G protein-linked transmembrane receptors may be expressed as a fusion protein. In other embodiments, G protein-linked transmembrane receptor protein is loaded into or onto the parent erythroid cell. In some embodiments, the loaded G protein-linked transmembrane receptors is additionally functionalized or otherwise modified.

In an embodiment, the receiver comprises a polypeptide that comprises an amino acid sequence derived from Duffy Antigen Receptor for Chemokines (DARC). In an embodiment, the functional parent erythroid cell expresses an exogenous gene encoding an amino acid sequence derived from Duffy Antigen Receptor for Chemokines (DARC). The DARC receiver may be expressed as a full-length protein or a fragment thereof. DARC may be expressed as a fusion protein. In other embodiments, DARC protein is loaded into or onto the parent erythroid cell. In some embodiments, the loaded DARC is additionally functionalized or otherwise modified. The DARC receiver molecule may be localized intracellularly or extracellularly. DARC was identified as a potent multi-ligand chemokine receptor. DARC belongs to the family of rhodopsin-like seven-helix transmembrane proteins. Besides erythrocytes DARC is expressed in post capillary venular endothelial cells, which are the primary site of leukocyte transmigration in most tissues. DARC provides a highly specific binding site for both CC and CXC chemokines. DARC is thought to possess a higher affinity for ELR motif CXC chemokines. CXC chemokines are neutrophil chemoattractants and may potentially be pro-angiogenic.

In an embodiment, the receiver comprises a polypeptide that comprises an amino acid sequence derived from an antibody. In one embodiment, the functional parent erythroid cell expresses an exogenous gene encoding an amino acid sequence derived from an antibody. The antibody receiver may be expressed as a full-length protein or a fragment thereof. The antibody may be expressed as a fusion protein. In other embodiments, the antibody protein is loaded into or onto the parent erythroid cell or directly into or onto the NV. In some embodiments, the loaded antibody is additionally functionalized or otherwise modified. The antibody receiver may be localized intracellularly or extracellularly. In one embodiment, the receiver comprises an antibody amino acid sequence that is specific for a desired target. In some embodiments, the antibody is a scFv. In other embodiments, the antibody is a nanobody.

In an embodiment, the receiver comprises a polypeptide that comprises an amino acid sequence derived from a scFv antibody.

In certain embodiments the receiver is capable of responding to an external stimulus, e.g., upon binding to a ligand or contacting the stimulus, wherein responding entails, for example, moving, re-folding, changing conformation, forming a dimer, forming a homodimer, forming a heterodimer, forming a multimer, transducing a signal, emitting energy in a detectable form (e.g., fluorescence), functionally interacting with another receiver, or functionally interacting with a non-receiver polypeptide.

In some embodiments, receivers comprise a protein-binding partner or a receptor on the surface of the NV, which functions to target the NV to a specific tissue space or to interact with a specific moiety on a recipient cell, either in vivo or in vitro. Suitable protein-binding partners include antibodies and functional fragments thereof, scaffold proteins, or peptides. Additionally, NVs can be employed to direct the synthesis and extracellular localization of lipids, carbohydrates, or other biological moieties.

In other embodiments, the interaction of the receiver with a target comprises altering the RNA composition of the target. In other embodiments, the interaction of the complex with a target comprises inducing translation in the target of a complex-associated RNA. In other embodiments, the interaction of the complex with a target comprises altering the expression of the target.

In some embodiments, the receiver may be engineered for specific trafficking from the parent cell to the NV. In some embodiments, the receiver or payload may be directed for trafficking by an addition of a molecule to the receiver (e.g. conjugation or fusion of another molecule). In certain embodiments the additional molecule may be appended via a linker.

In some embodiments, a nucleic acid receiver may be engineered for specific trafficking from the parent cell to the NV. In certain embodiments, a nucleic acid receiver (e.g., mRNA or miRNA) may comprise a sequence in the coding or noncoding region that targets the nucleic acid to the NV. In certain embodiments, the noncoding region may include a 3′ UTR or 5′ UTR.

Targets

A suitable receiver may be chosen to interact with a specific target. Suitable targets include entities that are associated with a specific disease, disorder, or condition. However, targets may also be chosen independent of a specific disease, disorder, or condition.

In certain embodiments, the NVs may interact with more than one type of target cell.

In some embodiments, the target is a circulating cell, an immune cell, an inflammatory cell, a tumor cell, or a metastatic cancer cell.

In some embodiments, the target is a mammalian cell, including but not limited to, for example, a human cell, a circulating cell, an immune cell, a neutrophil, an eosinophil, a basophil, a lymphocyte, a monocyte, a B cell, a T cell, a CD4+ T cell, a CD8+ T cell, a gamma-delta T cell, a regulatory T cell, a natural killer cell, a natural killer T cell, a macrophage, a Kupffer cell, a dendritic cell, or a cancer cell.

In certain embodiments, the target is a non-circulating cell or tissue.

In some embodiments, the target is an inflammatory molecule, a cytokine or a chemokine.

In some embodiments, the target is a bacterium.

In some embodiments, the target is a virus.

In some embodiments, the target is a parasite.

In some embodiments, the target is a fungus.

Indications

In certain aspects, the methods described herein are performed to prevent or treat an inflammatory related condition, inflammatory related disease or inflammatory related disorder. In certain aspects, the inflammatory related condition to be prevented or treated is known in the art to, in at least in a minority of cases, lead to increased inflammation in a subject harboring the inflammatory related condition. Inflammation may be contributing to the onset and/or the severity of the inflammatory related condition or disease. The inflammation may be a side-effect of the condition or disease or the inflammation may be a side-effect of another treatment for the condition, disorder or disease. Examples of inflammatory related conditions, disorders or diseases include, but are not limited to, asthma, sepsis, infection, Rheumatoid arthritis, ulcerative colitis, Crohn's disease, tuberculosis, hepatitis, sinusitis, autoimmune disease, inflammatory bowel disease, pelvic inflammatory disease, ulcers, atherosclerosis, erythema, necrosis, vasculitis, ankylosing spondylitis, connective tissue disease, kidney disease, sarcoidosis, thyroiditis, osteoarthritis, Rheumatism, chronic inflammatory demyelinating polyneuropathy, pancreatitis, psoriatic arthritis, periodontitis, Behcet's disease, sinusitis, polymyalgia rheumatic, nephritis, diverticulitis, granulomatosis with polyangilitis, granuloma, encephalitis, immune-mediated inflammatory disease, esophagitis, gout, uveitis, myopathy, gallbladder disease, periodic fever syndrome, interstitial cystitis, peritonitis, appendicitis, Parkinson's disease, Alzheimer's, systemic lupus erythematous, fibromyalgia, diverticulitis, dermatitis and ankylosing spondylitis. In some embodiments, the inflammatory related condition is an infection, for example, a bacterial infection, a viral infection, or a parasitic infection.

Rotes of Administration

In certain embodiments, the compositions described herein may be administered to into a subject and includes concurrent and sequential introduction of one or more compositions or agents (e.g., NVs). The introduction of the composition or agents described herein into a subject is by any suitable route, including orally, pulmonarily, intranasally, parenterally (intravenously, intramuscularly, intraperitoneally, or subcutaneously), rectally, intralymphatically, or topically. Administration includes self-administration and the administration by another. A suitable route of administration allows the composition or the agent to perform its intended function. For example, if a suitable route is intravenous, the composition is administered by introducing the composition or agent into a vein of the subject. Administration can be carried out by any suitable route. The compositions described herein can be administered in one or more administrations.

Dosing Schedules

In certain aspects, the amount of the compositions described herein that are administered to subjects, is an amount to achieve a therapeutic result (e.g., reduce inflammation in the subject). In certain embodiments, a therapeutically effective amount is administered that is sufficient to palliate, ameliorate, stabilize, reverse, slow or delay the progression of the disease state. Thus, the quantity of administration of the compositions and/or agents described herein (e.g., pharmaceutical compositions comprising NVs derived from MSCs) or the frequency of administration of a specific quantity of the compositions and/or agents is sufficient to reasonably achieve a desired therapeutic and/or prophylactic effect. For example, it may include an amount that results in the prevention of, treatment of, or a decrease in, the symptoms associated with a disease or condition that is being treated, e.g., the diseases or medical conditions associated with a target cell or target molecule. The amount of a therapeutic composition administered to the subject will depend on the type and severity of the disease and on the characteristics of the individual, such as general health, pathologic conditions, diets, age, sex, body weight and tolerance to drugs. It will also depend on the degree, severity and type of disease. Further, the effective amount will depend on the methods of formulation and administration used, e.g., administration time, administration route, excretion speed, and reaction sensitivity. The skilled artisan will be able to determine appropriate dosages depending on these and other factors. The compositions can also be administered in combination with one or more additional therapeutic compounds. A desirable dosage of the pharmaceutical composition may be in the range of about 0.001 to 100 mg/kg for an adult. In an example, an intravenous administration is initiated at a dose which is minimally effective, and the dose is increased over a pre-selected time course until a positive effect is observed. Subsequently, incremental increases in dosage are made limiting to levels that produce a corresponding increase in effect while taking into account any adverse effects that may appear. Non-limited examples of suitable dosages can range, for example, from 1×10⁷ to 1×10¹⁰, 1×10¹⁰ to 1×10¹⁴, from 1×10¹¹ to 1×10¹³, or from 5×10¹¹ to 5×10¹² NVs of the present invention. Specific examples include about 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², or more NVs of the present invention. Each dose of NVs can be administered at intervals such as once daily, once weekly, twice weekly, once monthly, or twice monthly.

Pharmaceutical Compositions

Methods for treatment of inflammatory related conditions and/or inflammatory related diseases are also encompassed by the present invention. Said methods of the invention include administering a therapeutically effective amount of a pharmaceutical composition comprising NVs. The NVs of the invention can be formulated in pharmaceutical compositions. These compositions can comprise, in addition to one or more of the distinct preparations of NVs, other drugs (e.g., anti-inflammatory agents), and a pharmaceutically acceptable excipient, carrier, buffer, stabiliser or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives can be included, as required.

In some embodiments, the NVs are prepared with carriers that will decrease the rate with which NVs are eliminated from the body of a subject. For example, controlled release formulation are suitable, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc.

In an embodiment the pharmaceutical composition comprising NVs is administered intravenously into a subject that would benefit from the pharmaceutical composition. In other embodiments, the composition is administered to the lymphatic system, e.g., by intralymphatic injection or by intranodal injection (see e.g., Senti et al., 2008 PNAS 105(46): 17908), or by intramuscular injection, by subcutaneous administration, by direct injection into the thymus, or into the liver.

In an embodiment, the pharmaceutical composition comprising NVs is administered as a liquid suspension. In one embodiment the pharmaceutical composition is administered as a coagulated formulation that is capable of forming a depot following administration, and in a preferred embodiment slowly release NVs into circulation, or in a preferred embodiment remain in depot form.

In an embodiment, the pharmaceutical composition comprising NVs is stored using methods and buffer compositions that are capable of maintaining viability of the NVs. For example, deoxygenation prior to storage to maintain an anaerobic state, manipulation of pH, supplementation of metabolic precursors, manipulation of osmotic balance, increasing of the volume of the suspending medium, and/or reduction of oxidative stress by adding protective molecules can be used to maintain the viability of the NVs. Several studies employing a combination of these strategies have reported maintenance of viability of erythrocytes allowing an extension of storage beyond 6 weeks (see e.g., Yoshida and Shevkoplyas, Blood Transfus 2010 8:220).

Pharmaceutically acceptable carriers or excipients may be used to deliver the NVs described herein. Excipient refers to an inert substance used as a diluent or vehicle. Pharmaceutically acceptable carriers are used, in general, with a compound so as to make the compound useful for a therapy or as a product. In general, for any substance, a pharmaceutically acceptable carrier is a material that is combined with the substance for delivery to a subject. Conventional pharmaceutical carriers, aqueous, powder or oily bases, thickeners and the like may be necessary or desirable. In some cases the carrier is essential for delivery, e.g., to solubilize an insoluble compound for liquid delivery; a buffer for control of the pH of the substance to preserve its activity; or a diluent to prevent loss of the substance in the storage vessel. In other cases, however, the carrier is for convenience, e.g., a liquid for more convenient administration. Pharmaceutically acceptable salts of the compounds described herein may be synthesized according to methods known to those skilled in the arts.

Typically, pharmaceutically acceptable compositions are highly purified to be free of contaminants, are biocompatible and not toxic, and are suited to administration to a subject. If water is a constituent of the carrier, the water is highly purified and processed to be free of contaminants, e.g., endotoxins.

The pharmaceutically acceptable carrier may be lactose, dextrose, sucrose, sorbitol, mannitol, starch, gum acacia, calcium phosphate, alginates, gelatin, calcium silicate, micro-crystalline cellulose, polyvinylpyrrolidone, cellulose, water, syrup, methyl cellulose, methylhydroxy benzoate, propylhydroxy benzoate, talc, magnesium stearate, and/or mineral oil, but is not limited thereto. The pharmaceutical composition may further include a lubricant, a wetting agent, a sweetener, a flavor enhancer, an emulsifying agent, a suspension agent, and/or a preservative.

Provided are pharmaceutical compositions containing NVs having effective levels of receivers. Such compositions contain a plurality of NVs, e.g., 1×10³ NVs, or 1×10⁴, 1×10⁵, 1×10⁶, 1×10⁷, 1×10⁸, 1×10⁹, 1×10¹⁰, 1×10¹¹, 1×10¹², or greater than 1×10¹² NVs. In specific examples, NVs generated from parent erythroid cells may be administered in a saline solution at a concentration of 10%, 20%, 30%, 40%, 50%, 60%, 70%, 80%, 90%, or greater than 90% mass to volume ratio (% m/v). The time of administration to a patient may range from 10 minutes to four hours, or more.

The pharmaceutically useful compound according to the present invention that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of the inflammatory related condition or inflammatory related disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980. A composition can be administered alone or in combination with other treatments, either simultaneously or sequentially dependent upon the condition to be treated.

The dosing and frequency of the administration of the NVs and pharmaceutical compositions thereof can be determined by the attending physician based on various factors such as the severity of disease, the patient's age, sex and diet, the severity of any inflammation, time of administration, and other clinical factors. In one example, an intravenous administration is initiated at a dose which is minimally effective, and the dose is increased over a pre-selected time course until a positive effect is observed. Subsequently, incremental increases in dosage are made limiting to levels that produce a corresponding increase in effect while taking into account any adverse effects that may appear.

Non-limited examples of suitable dosages can range, for example, from 1×10¹⁰ to 1×10¹⁴, from 1×10¹¹ to 1×10¹³, or from 5×10¹¹ to 5×10¹² NVs. Specific examples include about 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², or more NVs. Each dose of NVs can be administered at intervals such as once daily, once weekly, twice weekly, once monthly, or twice monthly.

“Complex-based proportional dosage” is the number of NVs administered as a dose relative to a naturally occurring quantity of circulating entities. The circulating entities may be cells, e.g., erythrocytes, reticulocytes, or lymphocytes, or targets, e.g., antigens, antibodies, viruses, toxins, cytokines, etc. The units are defined as NV per circulating entity, ie SCMRC/CE. This dosage unit may include 10⁻⁷, 10⁻⁶, 10⁻⁵, 10⁻⁴, 10⁻³, 10⁻², 10⁻¹, 1, 10, 10², 10³, 10⁴, 10⁵, 10⁶, 10⁷, 10⁸, or 10⁹.

The pharmaceutical compositions described herein comprise a NV and optionally a pharmaceutically active or therapeutic agent. The therapeutic agent can be a biological agent, a small molecule agent, or a nucleic acid agent.

Dosage forms are provided that comprise a pharmaceutical composition comprising a NV described herein. In some embodiments, the dosage form is formulated as a liquid suspension for intravenous injection.

Medical devices are provided that comprise a container holding a pharmaceutical composition comprising a NV described herein and an applicator for intravenous injection of the pharmaceutical composition to a subject.

Medical kits are provided that comprise a pharmaceutical composition comprising a NV described herein and a medical device for intravenous injection of the pharmaceutical composition to a subject.

A pharmaceutically acceptable suspension of NVs is preferably packaged in a volume of approximately 10 to approximately 250 ml. The packaging can be a syringe or an IV bag suitable for transfusions. Administration of the suspension is carried out, e.g., by intravenous or intra-arterial injection, optionally using a drip from an IV bag or the like. The administration is typically carried out intravenously in the arm or via a central catheter. For administrations exceeding 50 ml use of a drip is preferred.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B(1992).

### Example 1: Characterization of NVs

### Methods

Preparation of NVs:

Human primary bone marrow-derived MSCs were cultivated in alpha MEM medium supplemented with 15% fetal bovine serum (FBS, HyClone laboratories) and 1% antibiotic solution (100 units/ml penicillin and 100 mg/ml streptomycin, HyClone laboratories, Inc.) and used between the third and fifth passages. After 24 h of incubation in serum-free medium, the cells were washed with phosphate buffered saline (PBS, pH 7.4) and detached from the cell culture flasks using PBS containing 10 mM EDTA. MSCs were resuspended at a density of 2-5×10⁶ cells per mL in total 10 mL of PBS. Cell suspensions were passed five times through each of the polycarbonate membrane membrane filters (Whatman) with a pore size of 10 μm, 5 μm and 1 μm, in that order using a mini-extruder (Avanti Polar Lipids). Respectively 1 and 2 mL of 50 and 10% solution of iodixanol (Axis-Shield PoC AS), followed by 7 mL of the cell suspension effluent from the membrane filter were sequentially added to each 10 mL ultracentrifuge tube. The layers formed between 50% iodixanol and 10% iodixanol after ultracentrifugation at 100,000×g for 2 hours was collected and considered NVs.

Electron Microscopy:

Formvar/carbon-coated copper grids (Ted Pella, Inc., Redding, Calif., USA) were glow discharge-treated before the samples were loaded. The grids and samples were incubated for 15 minutes before being fixed in 2% paraformaldehyde and in 2.5% glutaraldehyde with PBS washes in between. Samples were washed in dH2O and then contrasted in 2% uranyl acetate. The preparations were examined using a LEO 912AB Omega electron microscope (Carl Zeiss NTS, Jena, Germany).

Results

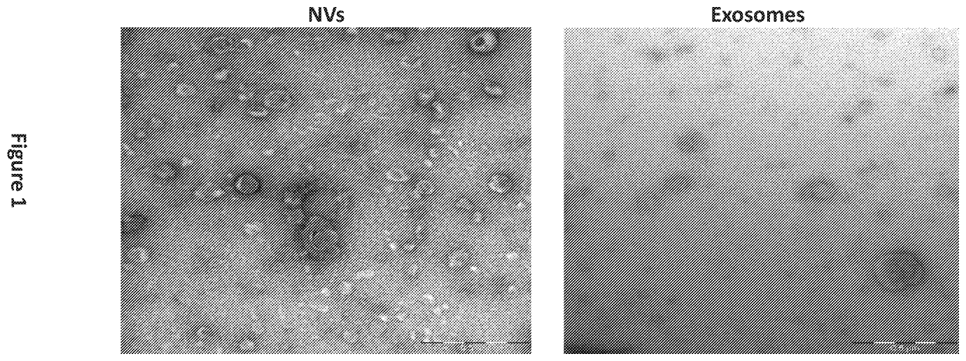

NVs were prepared from MSCs as described above. NVs and endogenous exosomes presented similar shape and size, as seen by transmission electron microscopy (FIG. 1).

### Example 2: RNA is Enriched in NVs Compared to Exosomes

### Methods

RNA and DNA Analysis:

RNA from exosomes or NVs was isolated using miRCURY™ RNA Isolation Kit for Biofluids (Exiqon) according to manufacturer's protocol. DNA was isolated using Qiamp DNA Blood Mini kit (Qiagen) according to manufacturer's protocol. One microliter of isolated RNA or DNA were analyzed for its quality, yield, and nucleotide length with capillary electrophoresis using Agilent RNA 6000 Picochip and Agilent High sensitivity DNA chip, respectively, on an Agilent 2100 Bioanalyzer® (Agilent Technologies).

### Results

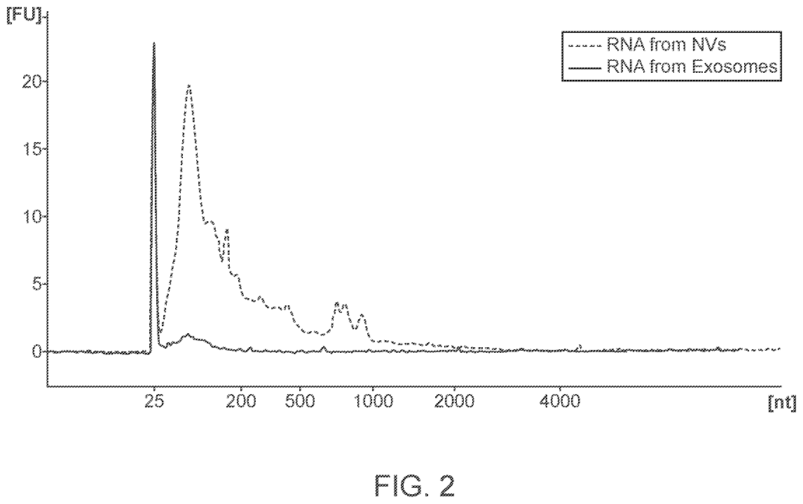

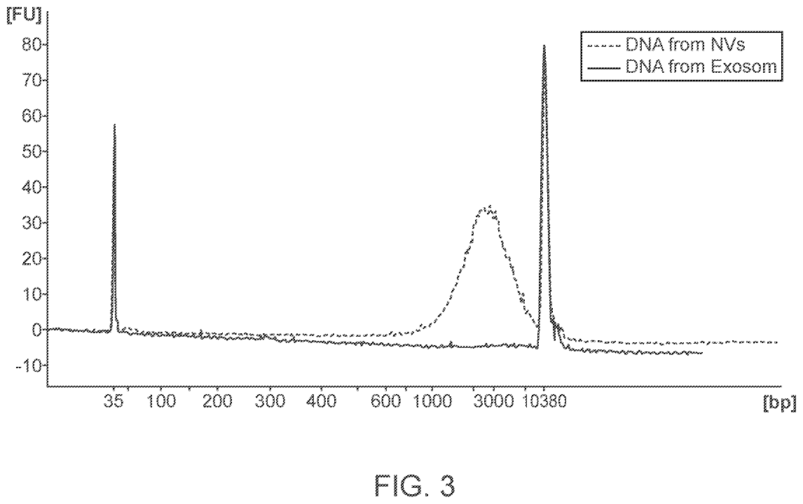

The total concentration of RNA was enriched in NVs in comparison to that of exosomes. Both types of vesicles presented a peak in concentration of RNA molecules below 200 nucleotides (FIG. 2). NVs also presented DNA, while the presence of DNA was undetectable in exosomes (FIG. 3).

### Example 3: Biodistribution of NVs in Mice

Methods

NVs were incubated with Cy7 monoNHS ester (5 μM, Amersham Biosciences) for 2 hours at 37° C. Cy7-labeled NVs were isolated using two-step Optiprep density gradient ultracentrifugation (10% and 50% iodixanol) at 100,000×g for 2 hours. Cy7-labeled NVs (8×10⁹) were administered intraperitoneally or intranasally to C57Bl/6 mice. After 30 minutes, 6 hours, and 24 hours, Cy7 fluorescence in the whole body of the mice was acquired by MS spectrum (Caliper Life Sciences). In addition, fluorescence in tissues including liver, heart, kidney, spleen, lung, pancreas and intestines was detected.

Results

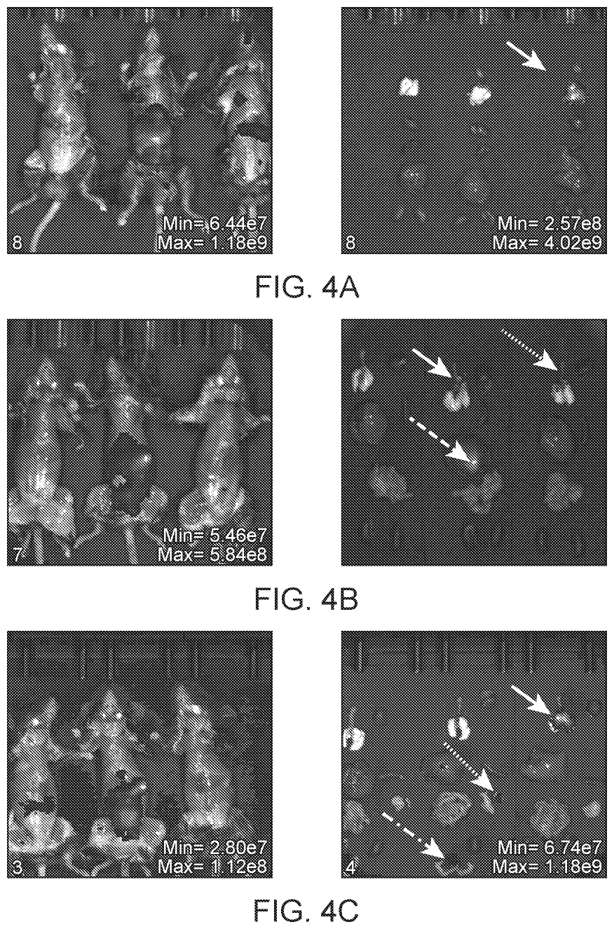

Thirty minutes after intranasal instillation, it was possible to observe that NVs reached the lungs (FIG. 4A, right), and the presence of NVs there was detected as long as 24 h after the instillation (FIG. 4C), while no vesicles were detected in other organs such as liver, kidneys, heart and spleen of those animals. Following intraperitoneal injection, despite a strong signal was detected in the abdomen of the recipient mouse after 30 min, no fluorescence was detected in collected organs (FIG. 4A, center). Six hours after instillation, it was possible to observe the presence of fluorescence in the liver and in lung lymphnodes of the animal that received the intraperitoneal injection (FIG. 4B). One day after the injection, nevertheless, the fluorescence was concentrated in the pancreas and bladder.

### Example 4: Anti-Inflammatory Effect of NVs in Mouse Model of Asthma

Methods

C57Bl/6 mice were sensitized to ovalbumin (through two intraperitoneal injections of 8 mg chicken ovalbumin (OVA) (Sigma-Aldrich) bound to 4 mg aluminium hydroxide (Sigma-Aldrich) in phosphate buffered saline (PBS) 5 days apart). The mice were then randomly divided into OVA (intranasally exposed to 100 μg OVA in 25 μL of PBS on 5 consecutive days) and control (C, exposed to PBS) groups. The mice were further randomized into groups that received 2×10⁹ exosomes or NVs, in 25 μL of PBS intranasally immediately following the first OVA or PBS exposure. The animals were sacrificed 24 h after the last OVA exposure, and bronchoalveolar lavage fluid was obtained (PloS One. 2011; 6 (5): e19889). Cystospins were made from BALF cells, stained with Giemsa and analyzed under a light microscope at 200× magnification.

For single-cell suspension from lung tissues, a fraction of the lung was collected, weighted and the tissue was mechanically disrupted using a Miltenyi GentleMacs™ Dissociator, and subsequently digested with 1 mg/mL of collagenase D (Roche) and 40 U/mL of DNase I. The cells were collected, and incubated with antibodies against the following surface markers: CD3—conjugated with FITC, B220—conjugated with PE, CD45—conjugated with APC, CD4—conjugated with APC-H7, CD25—conjugated with BV421 (all purchased from BD Biosciences). The cells were analyzed using a FACS Verse Flow Cytometer (BD Biosciences) running BD FACSuite Software and analyzed with FlowJo Software 1l (TreeStar Inc.). The levels of Eotaxin-2 in the BALF supernatant and in lung tissue lysates were determined using a commercial ELISA kit (R&D Systems). All statistical analyses were performed using the software Prism v8 (GraphPad).

Results

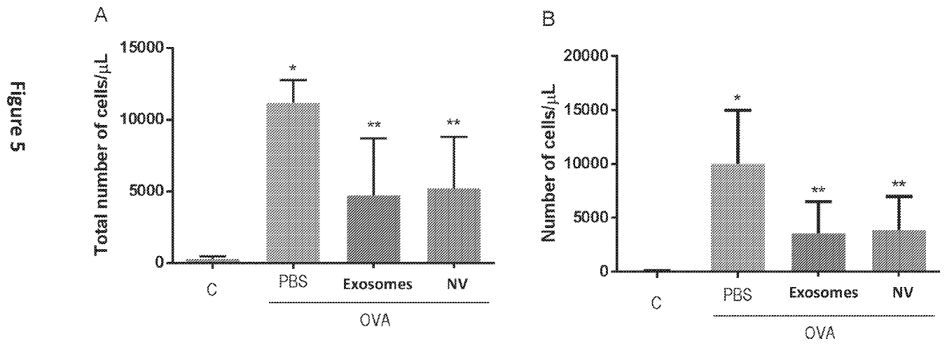

Local administration of NVs derived from human bone-marrow derived MSC reduced the cellularity and number of eosinophils in bronchoalveolar lavage fluid (BALF) of OVA sensitized and exposed animals, as comparable to the administration of naturally produced exosomes from the same cells (FIG. 5). In addition, NV treatment resulted in decreased number of inflammatory cells within the lung tissue (FIG. 6), which was associated with lower levels of CCL24/eotaxin-2 in both BALF and lung tissue (FIG. 7).

### Example 5: Anti-Inflammatory Effect of NVs in Bacterial Outer Membrane Vesicles-Induced Actue Inflammation

Methods

Bacteria outer membrane vesicles (OMVs) have been considered as infectious agents to induce inflammation (Nat Commun. 2016 Jan. 25; 7:10515). RAW 264.7 (1×10⁵), a mouse macrophage cell line, were seeded into 24-well plates. OMVs (100 ng/mL) were applied to RAW 264.7 to induce pro-inflammatory cytokines (TNF-α and IL-6) for 3 hours, followed by addition of various numbers (1×10⁹, 2×10⁹, and 3×10⁹) of NVs. Supernatant concentrations of TNF-α and IL-6 were measured by ELISA kit (R&D systems) at 15 hours.

Results

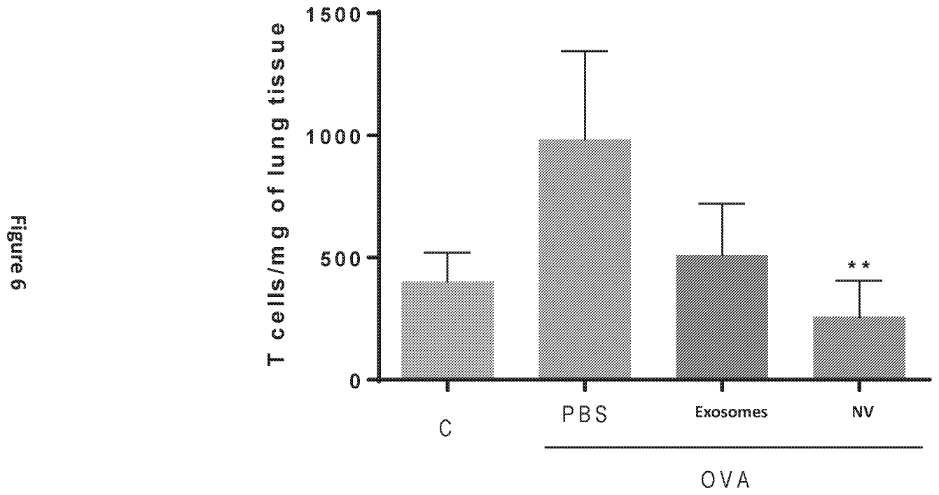

NVs reduced significantly OMVs-induced release of TNF-α and IL-6 from RAW 264.7 cells, revealing an anti-inflammatory role of NVs derived from MSCs (FIG. 8).

### Example 6: Treatment of Sepsis by Administration of NVs Comprising Inhibitor of Myd88

### Methods

To confirm the therapeutic effect of NVs comprising inhibitors of Myd88 for the treatment of sepsis, NVs are prepared as described above in Example 1 and loaded with either siRNAs targeted against Myd88, or a synthetic peptide inhibitor of Myd88. NVs are administered intravenously to a murine model of sepsis. Two murine models of sepsis are tested: the cecal ligation and puncture (CLP) model and the lipopolysaccharide (LPS) induced model. For the CLP model, the mice are randomly divided into CLP mice and control groups. The CLP procedure is preformed and the mice are monitored for development of symptoms of sepsis (e.g., body temperature, heart rate and respiratory rate) and/or blood sample collected for testing for indicators of sepsis (e.g., pro-inflammatory cytokine levels such as TNF-α or Interleukin-1β) 1 h to 24 h after the CLP procedure is performed. For the LPS-induced model of sepsis, mice were intraperitoneally injected with 10 mg/kg LPS and blood is collected at 1 h to 24 h after LPS challenge to test for induction of symptoms or markers of sepsis. The mice were further randomized into groups that receive 2×10⁹ exosomes or NVs, in 25 μL of PBS intranasally. Mice are regularly monitored for symptoms of sepsis or markers of sepsis from blood samples. The animals are sacrificed 24 h to one week after administration of the exosomes or NVs, and blood samples are obtained for analysis.

### Example 7: Anti-Inflammatory Effect of MSC-NVs in Bacterial Outer Membrane Vesicles (OMVs)-Induced Sepsis In Vivo

### Methods

Mice (wild-type mice of the C57BL/6 genetic background; 6 weeks old) were intraperitoneally (i.p.) injected with OMVs (15 μg) to provoke sepsis as previously described [1, 2], and then administrated with NVs (2×10⁹) i.p. Mice were sacrificed at 6 h following anesthetization with i.p injection of xylazine chloride (10 mg/kg; Bayer) and ketamine hydrochloride (100 mg/kg). Rectal temperature was measured by thermometer (Bioseb). Peritoneal fluid (PF), blood, and bronchoalveolar lavage fluid (BALF) were collected from mice, and then the supernatants were stored at −80° C. for cytokine analysis following centrifugation. The pelleted cells were analyzed using flow cytometry and light microscopy. For analysis of surface marker expression, freshly isolated cells from peritoneum were stained with fluorochrome conjugated antibodies. Viable cells were blocked for non-specific staining with 2.4G2 (anti-Fc-receptor) and stained with Ly6G BV650 and Ly6C BV605 (BD Biosciences). To exclude dead cells, 7-Aminoactinomycin D (Sigma Aldrich) stained positive cells were excluded from the analysis as shown in the gating strategy. Events were collected and analyzed by using an LSR-II Flow cytometer (BD Biosciences) and FlowJo software (Tree Star Inc.). For counting leukocytes and platelets in blood, blood samples were obtained by cardiac puncture, followed by transferring the blood into EDTA-tubes. The number of leukocyte and platelets were counted using the light microscopy following incubation with 1% hydrochloride and Rees-Ecker diluting fluid (Thermo Fisher Scientific), respectively. The concentrations of cytokines in serum were measured using DuoSet ELISA Development kit (R&D Systems). Neutralizing antibody experiments used rat IgM anti-mouse IL-10 mAb (200 g per body; U-CyTech Biosciences) or an isotype control antibody (Thermo Fisher Scientific).

Results

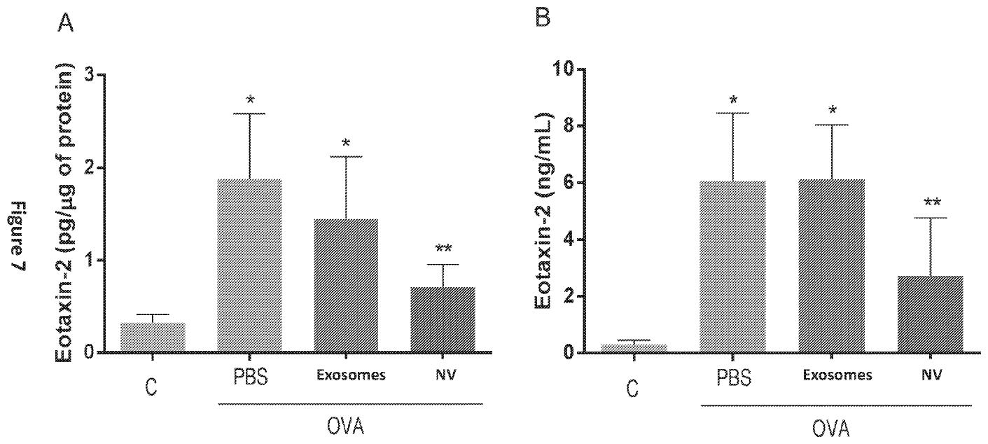

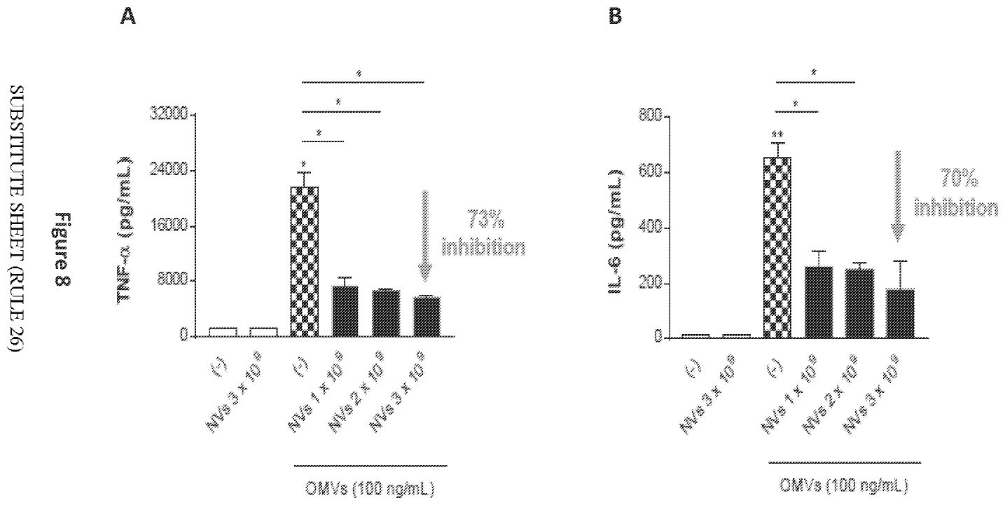

To address whether MSCs-derived NVs contribute to immunosuppression in the mouse sepsis model, a sublethal dose of E. coli OMVs (15 μg) was firstly injected i.p. once to establish septic mice. NVs (2×10⁹) were then injected i.p. once at 1 h followed by analysis at 6 h to monitor the anti-inflammatory response, as shown in FIG. 9A. OMVs-treated animals showed usual eye exudates, but this symptom was abrogated following treatment with NVs (FIG. 9B). Moreover, a decreased pattern in body temperature and weight commonly observed in the OMVs-induced sepsis model [3], was recovered by treatment with NVs at 6 h (FIGS. 9C and 9D).

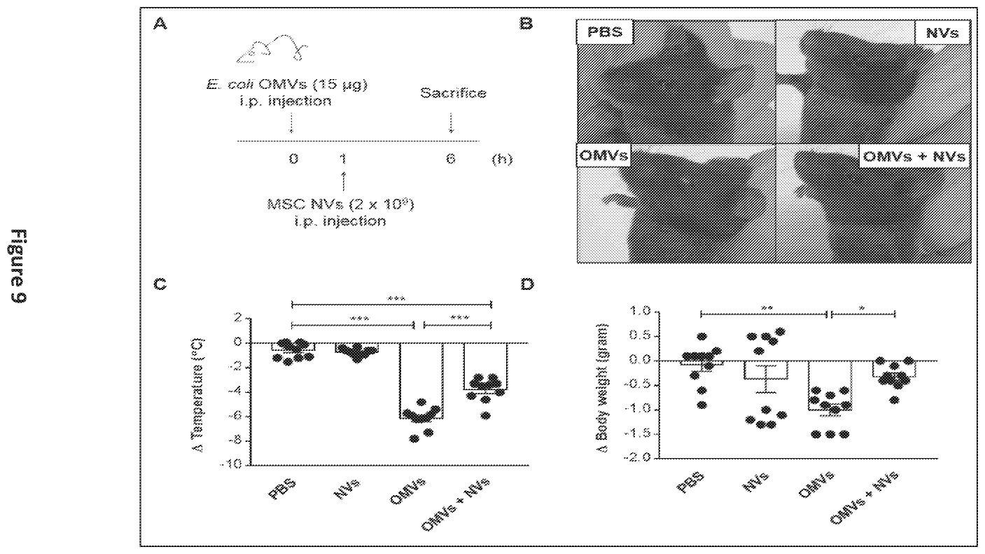

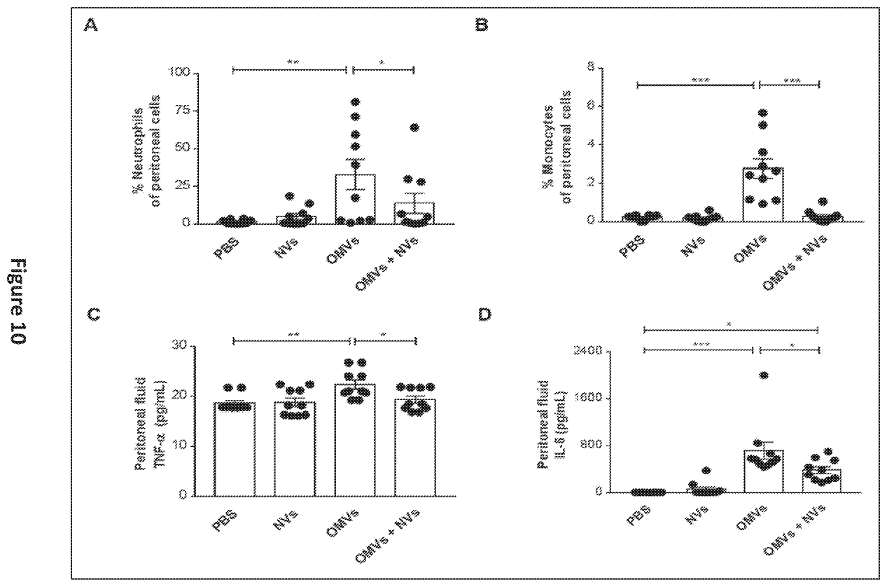

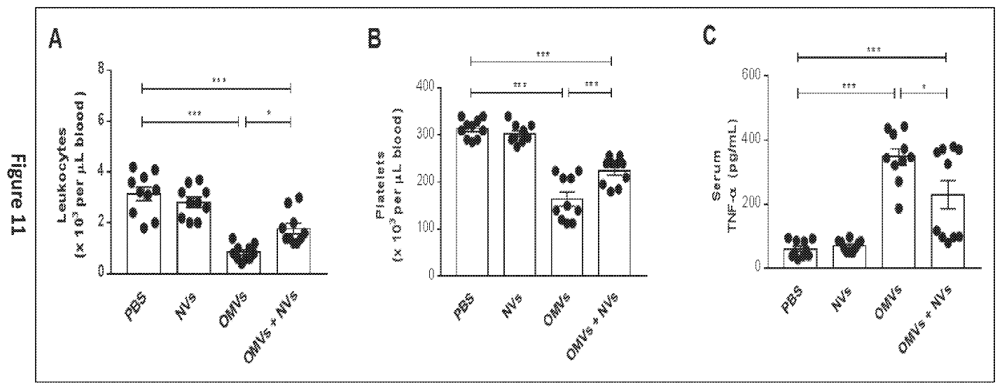

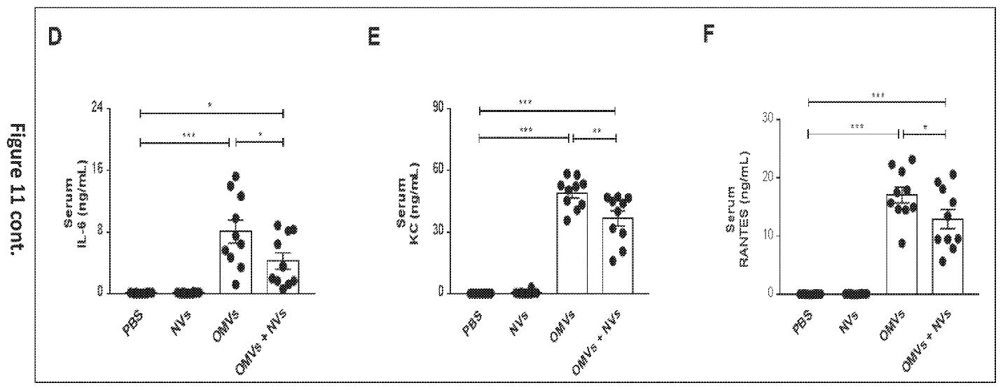

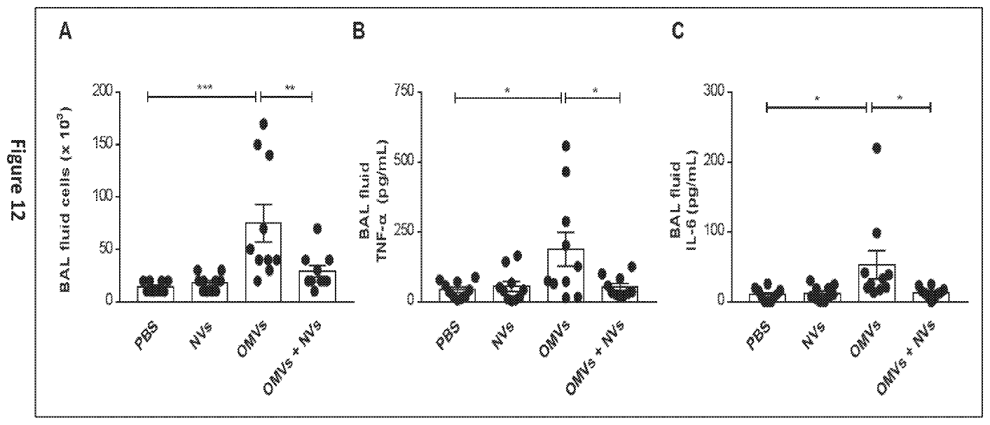

Intraperitoneal administration of NVs reduced the number of neutrophil and monocyte infiltration in the peritoneum (FIGS. 10A and 10B) together with dampened pro-inflammatory cytokines responses for TNF-α and IL-6 (FIGS. 10C and 10D). In addition, NVs treatment recovered OMVs-triggered leukopenia and thrombocytopenia in blood (FIGS. 11A and 11B), and diminished induction of a variety of systemic cytokines and chemokine levels in serum (FIGS. 11C-11F). Moreover, NVs could modulate inflammatory responses of distant organs, such as lung (FIG. 12).

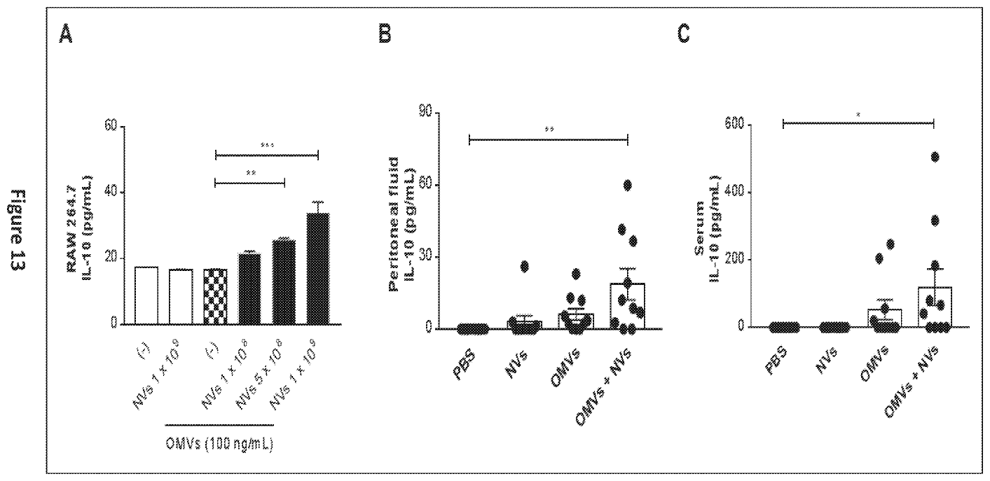

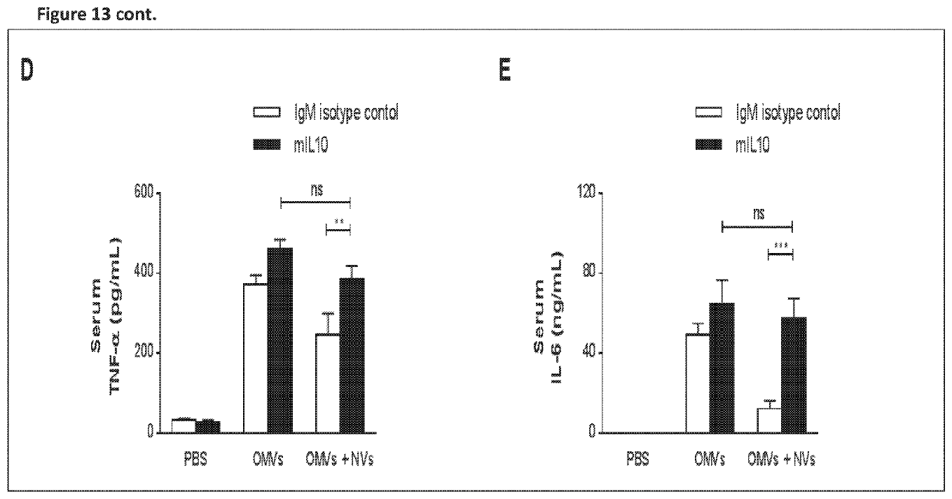

Given that Interleukin 10 (IL-10) has been associated with an effective anti-immune response [4], we next determined whether IL-10 affects NVs-induced anti-inflammation in sepsis. NVs could increase significantly the release of IL-10 from RAW 264.7 cells (FIG. 13A) as well as mouse model (FIGS. 13B and 13C). In addition, upon addition of IL-10-neutralizing antibody, the aforementioned protective effects of NVs in mice were lost (FIGS. 13D and 13E), suggesting that NVs-induced beneficial effects during sepsis may be through IL-10-mediated anti-inflammation.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### REFEERENCES CITED

[1] Small. 2015 Jan. 27; 11(4):456-61.
[2] Sci Rep. 2017 Dec. 12; 7(1):17434.
[3] PLoS One. 2010 Jun. 28; 5(6):e11334.
[4] Crit Care Med. 2002 January; 30(1 Suppl):S58-63.

1. A pharmaceutical composition for the prevention or treatment of inflammatory-related conditions comprising extracellular vesicle mimetic nanovesicles (NVs) derived from mesenchymal stem cells (MSCs) produced by the process of passing a suspension of MSCs through a plurality of membrane filters.

2. The pharmaceutical composition of claim 1, wherein the membrane filters comprise pores with diameters of 10 μm or less.

3. The pharmaceutical composition of claim 1 or 2, wherein the NVs have a diameter of between about 20 nm and 250 nm.

4. The pharmaceutical composition of any one of claim 1-3, wherein the NVs have a substantial amount of nucleic acid within the NVs.

5. The pharmaceutical composition of claim 4, wherein the nucleic acid within the NVs is RNA.

6. The pharmaceutical composition of any one of claims 1-5, wherein the MSCs are mammalian.

7. The pharmaceutical composition of any one of claims 1-6, wherein the MSCs are cultured in vitro.

8. The pharmaceutical composition of any one of claims 1-7, wherein the MSCs are primary cells.

9. The pharmaceutical composition of any one of claims 1-8, wherein the MSCs are derived from an autologous donor.

10. The pharmaceutical composition of any one of claims 1-9, wherein the inflammatory related condition is selected from the group consisting of asthma, sepsis, infection, Rheumatoid arthritis, ulcerative colitis, Crohn's disease, tuberculosis, hepatitis, sinusitis, autoimmune disease, inflammatory bowel disease, pelvic inflammatory disease, ulcers, atherosclerosis, erythema, necrosis, vasculitis, ankylosing spondylitis, connective tissue disease, kidney disease, sarcoidosis, thyroiditis, osteoarthritis, Rheumatism, chronic inflammatory demyelinating polyneuropathy, pancreatitis, psoriatic arthritis, periodontitis, Behcet's disease, sinusitis, polymyalgia rheumatic, nephritis, diverticulitis, granulomatosis with polyangilitis, granuloma, encephalitis, immune-mediated inflammatory disease, esophagitis, gout, uveitis, myopathy, gallbladder disease, periodic fever syndrome, interstitial cystitis, peritonitis, appendicitis, Parkinson's disease, Alzheimer's, systemic lupus erythematous, fibromyalgia, diverticulitis, dermatitis and ankylosing spondylitis.

11. The pharmaceutical composition of claim 10, wherein the inflammatory related condition is asthma.

12. The pharmaceutical composition of claim 10, wherein the inflammatory related condition is sepsis.

13. The pharmaceutical composition of claim 10, wherein the inflammatory related condition is infection.

14. The pharmaceutical composition of claim 12, wherein the infection is a bacterial, viral or parasitic infection.

15. The pharmaceutical composition of any one of the above claims, where upon administration to a subject, inflammation in at least one tissue in the subject is reduced.

16. The pharmaceutical composition of any one of the above claims, wherein the NVs are immunomodulatory when administered intravenously or non-parenterally to a human subject.

17. The pharmaceutical composition of any one of the above claims, where upon administration of the pharmaceutical composition results in reduced infiltration of T lymphocytes in lung tissue compared to administration of a pharmaceutical composition comprising the same concentration of exosomes derived from MSCs.

18. The pharmaceutical composition of any one of the above claims, further comprising a therapeutic payload.

19. The pharmaceutical composition of claim 18, wherein the therapeutic payload is an inhibitor of Myeloid Differentiation Factor 88 (Myd88).

20. The pharmaceutical composition of claim 19, wherein the inhibitor of Myd88 is a Myd88 synthetic peptide.

21. The pharmaceutical composition of claim 18, wherein the therapeutic payload is an inhibitor of GATA-3.

22. The pharmaceutical composition of any one of claims 18-21, wherein the therapeutic payload comprises nucleic acid.

23. The pharmaceutical composition of claim 22, wherein the therapeutic payload comprises RNA.

24. The pharmaceutical composition of any one of claims 18-21, wherein the therapeutic payload comprises a small-molecule inhibitor.

25. The pharmaceutical composition of any one of claims 18-21, wherein the therapeutic payload comprises an antibody.

26. The pharmaceutical composition of any one of the above claims, further comprising a targeting receiver.

27. The pharmaceutical composition of any one of the above claims, wherein the targeting receiver comprises a protein or peptide.

28. The pharmaceutical composition of any one of the above claims, wherein the pharmaceutical composition further comprises Interleukin 10 (IL-10).

29. The pharmaceutical composition of claim 28, wherein the NVs comprise IL-10.

30. The pharmaceutical composition of claim 29, wherein the IL-10 is displayed on the surface of the NVs.

31. The pharmaceutical composition of any one of the above claims, wherein the pharmaceutical composition is administered in conjunction with at least one additional therapeutic agent.

32. The pharmaceutical composition of claim 31, wherein the pharmaceutical composition is administered prior to the at least one additional therapeutic agent.

33. The pharmaceutical composition of claim 31, wherein the pharmaceutical composition is administered after the at least one additional therapeutic agent.

34. The pharmaceutical composition of claim 31, wherein the pharmaceutical composition is administered simultaneously with the least one additional therapeutic agent.

35. The pharmaceutical composition of any one of the above claims, wherein the pharmaceutical composition is administered intravenously.

36. The pharmaceutical composition of any one of the above claims, wherein the pharmaceutical composition is administered subcutaneously.

37. The pharmaceutical composition of any one of claims 31-36, wherein the at least one additional therapeutic agent is IL-10.

38. A method of treating or preventing an inflammatory-related condition, comprising administering an effective amount of the pharmaceutical composition of any one of the above claims.

39. The method of claim 38, wherein the inflammatory related condition is asthma.

40. The method of claim 38 wherein the inflammatory related condition is sepsis.

41. A method of treating acute inflammation, comprising administering an effective amount of the pharmaceutical composition of any one of claims 1-37.

42. A pharmaceutical composition for the treatment of asthma comprising NVs derived from MSCs, wherein administration of the pharmaceutical composition results in reduced infiltration of T lymphocytes in the lungs of subjects with asthma compared to administration of a pharmaceutical composition comprising the same concentration of exosomes derived from MSCs.

43. A method of preparing a pharmaceutical composition comprising a heterogeneous population of NVs for the prevention or treatment of inflammatory-related condition, the method comprising:
providing a plurality of donor MSCs from a human subject;
suspending the donor MSCs in a solution to create a MSC cell suspension;
passing the suspension of MSCs through a plurality of membrane filters, thereby generating a heterogeneous population of NVs.

44. The method of claim 43, wherein the membrane filters have comprise pores with diameters of 10 μm or less.

45. The method of claims 43 or 44, wherein the NVs have a diameter of between about 20 nm and 250 nm.

46. The method of any one of claims 43-45, wherein the NVs have a substantial amount of nucleic acid within the NVs.

47. The method of any one of claims 43-46, wherein the MSCs are human.

48. The method of any one of claims 43-47, wherein the MSCs are derived from an autologous donor.

49. The method of any one of claims 43-48, wherein the inflammatory related condition is selected from the group consisting of asthma, sepsis, infection, Rheumatoid arthritis, ulcerative colitis, Crohn's disease, tuberculosis, hepatitis, sinusitis, autoimmune disease, inflammatory bowel disease, pelvic inflammatory disease, ulcers, atherosclerosis, erythema, necrosis, vasculitis, ankylosing spondylitis, connective tissue disease, kidney disease, sarcoidosis, thyroiditis, osteoarthritis, Rheumatism, chronic inflammatory demyelinating polyneuropathy, pancreatitis, psoriatic arthritis, periodontitis, Behcet's disease, sinusitis, polymyalgia rheumatic, nephritis, diverticulitis, granulomatosis with polyangilitis, granuloma, encephalitis, immune-mediated inflammatory disease, esophagitis, gout, uveitis, myopathy, gallbladder disease, periodic fever syndrome, interstitial cystitis, peritonitis, appendicitis, Parkinson's disease, Alzheimer's, systemic lupus erythematous, fibromyalgia, diverticulitis, dermatitis and ankylosing spondylitis.

50. The method of any one of claims 43-49, wherein the inflammatory related condition is asthma.

51. The method of any one of claims 43-49, wherein the inflammatory related condition is sepsis.
